(19) United States
(12) Patent Application Publication
(10) Pub. No.: US 2017/0042870 A1
(43) Pub. Date: Feb. 16, 2017

(54) TARGETED NANOPARTICLE COMPOSITIONS AND METHODS OF THEIR USE TO TREAT OBESITY

(71) Applicants: THE BRIGHAM AND WOMEN'S HOSPITAL, INC., Boston, MA (US); MASSACHUSETTS INSTITUTE OF TECHNOLOGY, Cambridge, MA (US)

(72) Inventors: Yuan Xue, Livingston, NJ (US); Xiaoyang Xu, Fair Lawn, NJ (US); Xueqing Zhang, Fair Lawn, NJ (US); Robert S. Langer, Newton, MA (US); Omid C. Farokhzad, Waban, MA (US)

(21) Appl. No.: 15/119,336

(22) PCT Filed: Feb. 13, 2015
(86) PCT No.: PCT/US2015/015911

§ 371 Date: Aug. 16, 2016

Related U.S. Application Data
(60) Provisional application No. 61/940,732, filed on Feb. 17, 2014.

(51) Int. Cl. A61K 31/4439; A61K 9/51; A61K 47/48

(52) U.S. Cl. CPC A61K 31/4439; A61K 47/48915; A61K 47/48246; A61K 47/489; A61K 9/5153

(57) ABSTRACT

Nanoparticles having a positive feedback delivery system include an agent specific for a target in combination with a target inducing agent. Upon administration to a subject, the targeting moiety on the nanoparticles binds to available targets in the subject. The nanoparticles release the target inducing agent and, optionally, a therapeutic agent, at the site where the nanoparticles bind the target. The inducing agent causes additional targets to be expressed. More nanoparticles bind to the additional, induced targets. By inducing additional targets to be expressed at specific regions in the subject that require treatment, more nanoparticles can bind to the targets in that specific region of interest, increasing the concentration of nanoparticles at a specific area of subject is increased.

### STATEMENT REGARDING FEDERALLY SPONSORED RESEARCH

This invention was made with government support under Grants NIH/NIBIB R01 EB015419-01, NIH/NCI U54 CA1151884, NIH/NHLBI HSSN268201000045C, and NIH/NCI 1F32CA168163-02 awarded by the National Institutes of Health. The government has certain rights in the invention.

### FIELD OF THE INVENTION

The invention is generally directed to targeted nanoparticle compositions for treatment of obesity.

### BACKGROUND OF THE INVENTION

The incidence of obesity has increased at an astonishing rate in the past few decades in the world and obesity-related diseases have become a major threat to human health. According to the American Heart Association, the total excess cost in 2013 related to the current prevalence of adolescent overweight and obesity is estimated to be $254 billion ($208 billion in lost productivity secondary to premature morbidity and mortality and $46 billion in direct medical costs). If current trends in the growth of obesity continue, total healthcare costs attributable to obesity could reach $861 to $957 billion by 2030, which would account for 16% to 18% of US health expenditures (Statistical Fact Sheet 2013 Update, American Heart Association).

Current obesity therapeutic approaches include restriction of food intake, enhanced exercise, medication and plastic surgery. Limited clinical therapeutic agents are available for treating obesity due to a complex interplay among genetic, environmental and cultural factors. In addition, numerous weight-loss drugs have been abandoned because of undesired side effects. One of the top reasons is the drugs have broad targeting spectrums that affect multiple organs and tissues. Therefore, the targeted delivery of therapeutic agents to adipose tissue is important.

Therefore, it is an object of the invention to provide targeted compositions for the delivery of therapeutic agents to specific regions or areas in a subject, especially adipose tissue.

It is another object of the invention to provide methods of treating cell-specific diseases or disorders.

It is another object of the invention to provide compositions and methods of treating tissue-specific diseases or disorders.

It is another object of the invention to provide compositions and methods of treating organ-specific diseases or disorders.

### SUMMARY OF THE INVENTION

A multimodal nanoparticle platform technology that enables targeted drug delivery and, optionally, imaging, has been developed. The nanoparticles possess excellent stability, high loading efficiency, multiple agent encapsulation, targeting and, optionally, imaging. In a preferred embodiment, multimodal nanoparticles have three main components: 1) a targeting moiety (peptides, antibodies, small molecules, aptamers, etc.) that binds to a unique molecular signature on cells, tissues, or organs of the body; 2) an outside stealth layer that allows the particles to evade recognition by immune system components and increase particle circulation half-life; and 3) a biodegradable polymeric material, forming an inner core which can carry therapeutic payloads and release the payloads at a sustained rate after systemic, intraperitoneal, oral, pulmonary, or topical administration. Lipid may be incorporated into the nanoparticle as a conjugate to the polymer between the inner and outer layers or dispersed therein. The nanoparticles also optionally include a detectable label such as a fluorophore or NMR contrast agent that allows visualization of nanoparticles, for example, within plaques.

In one embodiment, nanoparticles have a positive feedback delivery system. For example, the drug-loaded nanoparticles include an agent specific for a target in combination with a target inducing agent. Upon administration to a subject, the targeting moiety on the nanoparticles binds to available targets in the subject. The nanoparticles release the target inducing agent and, optionally, a therapeutic agent, at the site where the nanoparticles bind the target. The inducing agent causes additional targets to be expressed. More nanoparticles bind to the additional, induced targets. By inducing additional targets to be expressed at specific regions in the subject that require treatment, more nanoparticles can bind to the targets in that specific region of interest. Thus, the concentration of nanoparticles at a specific area of subject is increased. This increase nanoparticle concentration at a specific region leading to an increase in the concentration of drug released from the nanoparticles, thereby amplifying the delivery of the drug to specific regions.

The nanoparticles can be loaded with a variety of therapeutic agents. For example, siRNAs or functional proteins such as FGF21 can be loaded within the targeted nanocarriers to regulate adipose tissue transformation and angiogenesis. Alternatively, it is possible to encapsulate multiple therapeutic agents into the same NP, which may offer additional effects in combination therapy.

Still another embodiment provides a method of preventing or treating one or more symptoms of a disease or disorder in a subject by administering the drug-loaded nanoparticles targeted to a specific cell, tissue, or organ in a subject to deliver the drug in combination with a target inducing agent. Representative diseases and disorders include metabolic disorders such as diabetes, obesity and/or obesity-associated disorders, cancer, and inflammation. For example, the nanoparticles can specifically deliver therapeutic compounds or imaging agents to specific tissue, for example, to white adipose tissue (WAT). Massive expansion of adipose tissues such as WAT leads to obesity, which has become a major threat to human health throughout the world. The inducing agent can be an agent that induces the transformation of WAT to brown adipose tissue (BAT). Another embodiment utilizes drug-loaded targeted nanoparticles that upregulate angiogenic and BAT markers, activate angiogenesis as represented by markedly intensified vascularization, and facilitate the transformation of WAT into brown-like adipose tissue.

Yet another embodiment provides a method for delivering a therapeutic agent to a region of interest in a subject in need thereof by administering a nanoparticle loaded with the therapeutic agent, for example, a drug, wherein the nanoparticle specifically binds to a target in the region of interest in the subject. The targeted nanoparticle also includes an agent that induces expression of the target in the subject. The delivery of the therapeutic agent to the region of interest increases over time due to the increase in binding of additional nanoparticles to newly induced targets.

### BRIEF DESCRIPTION OF THE DRAWINGS

FIG. 1A is a schematic presentation of the white adipose tissue (“WAT”) browning process through a positive feedback drug delivery system. Released Rosi and PGE2 promote transformation of WAT into brown-like adipose tissue and stimulate angiogenesis. This facilitates the homing of targeted NPs to adipose angiogenic vessels, thereby amplifying their delivery and hence expediting the WAT browning process. FIG. 1B shows the chemical structure of PLGA-b-PEG-Peptide/Rosiglitazone NPs (NPs). The particle consists of two components: i) an outer PEG surface with a targeting peptide; ii) a PLGA hydrophobic core which plays two roles: a) acting as a polymer matrix loaded with Rosiglitazone, b) promoting Rosiglitazone molecule retention inside the NP core and controlling drug release. Non-targeted and targeted NPs encapsulating Rosiglitazone were formulated via a single step emulsion method. FIG. 1C is a line graph of accumulated drug release (%) versus time (hours) showing the in vitro release profile of Rosiglitazone from NP-Rosi and iRGD-NP-Rosi. FIG. 1D is a histogram of intensity (%) versus diameter (nm) showing size distribution of the iRGD-NP-Rosi measured by dynamic light scattering.

FIG. 2A is bar graph showing relative expression levels of Integrinαv in stromal vascular fragments (SVF) from inguinal WAT treated with free Rosi (Rosie) and NP-encapsulated Rosi (NP-Rosi) by qRT-PCR (n=3 per group). NT represents no-treated control. FIG. 2B is a bar graph showing relative expression levels of Integrinα3 in SVF from inguinal WAT treated with free Rosi (Rosie) and NP-encapsulated Rosi (NP-Rosi) measured by qRT-PCR PCR (n=3 per group). NT represents non treated control. FIG. 2C is a bar graph showing proliferation (A₄₉₀) of SVF from inguinal WAT induced by Rosi (Rosi) and NP-Rosi treatments (NP-Rosi) measured with PCR (n=6 per group).

FIG. 3A is a bar graph of the density of CD31⁺ blood vessel area per optical field (×10³ μm²) quantified from confocal images. Data represent means±SEM from 9 samples from four mice in each group. Wild Type (WT), treated with free Rosi (Rosiglitazone) and NP-encapsulated Rosi (NP-Rosi) iRGD targeted NP loaded with Rosi (iRGD-NP-Rosi) and P3 targeted NP loaded with Rosi (P3-NP, Rosi). FIG. 3B is a bar graph of the average size of adipocyte (μm²) from different treatment groups: Wild Type (WT), treated with free Rosi (Rosi) and NP-encapsulated Rosi (NP-Rosi) iRGD targeted NP loaded with Rosi (iRGD-NP-Rosi) and P3 targeted NP loaded with Rosi (P3-NP, Rosi). Data represent means±SEM from 9 samples from four mice in each group. FIG. 3C is a bar graph of the numbers of isolectin-positive B4⁺ vessels per adipocyte per field. Wild Type (WT), treated with free Rosi (Rosi) and NP-encapsulated Rosi (NP-Rosi) iRGD targeted NP loaded with Rosi (iRGD-NP-Rosi) and P3 targeted NP loaded with Rosi (P3-NP, Rosi) Data represent means±SEM from 9 samples from four mice in each group.

FIG. 4A is a bar graph of relative expression level of Ucp1 in inguinal WAT in not treated (NT), free Rosi (Rosi), control CTRL, iRGD-NP-Rosi, and P3-NP-Rosi. FIGS. 4B-F are similar bar graphs showing expression levels CIDEA, DIO2, VEGFR2, early stage VEGF, and late stage VEGF, respectively, quantified by qRT-PCR. Data are means±SEM from three to four mice in each group.

FIG. 5 DIO mice received the treatments with Rosi, NP-Rosi, iRGD- and P3-NP-Rosi for 25 days. FIG. 5A is a photograph of representative mice at the experiment end point. FIG. 5B is a line graph of relative body weight increase (%) versus days after treatment of non-treated mice or mice receiving Rosi or NP-Rosi. Data are relative means±SEM from three to four mice in each group. FIG. 5C is a line graph of relative body weight increases (%) versus day after of non-treated mice or mice receiving iRGD-NP-Rosi or P3-NP-Rosi. Data are relative means±SEM from three to four mice in each group. FIG. 5D is a line graph of food intake (g) versus days after treatment per mouse per day. Data are means±SEM from three to four mice in each group. FIG. 5E is a line graph of CD31⁺ area/field (×10² μm²) of non-treated mice or mice receiving iRGD-NP-Rosi or P3-NP-Rosi. FIG. 5F is a line graph of relative expression level of Ucp1 of inguinal WATs from DIO mice treated with Rosi, NP-Rosi, iRGD-NP-Rosi and P3-NP-Rosi. Data are means±SEM from three to four mice in each group. FIG. 5G is a line graph of relative expression level of Vegfr2 of inguinal WATs from DIO mice treated with Rosi, NP-Rosi, iRGD-NP-Rosi and P3-NP-Rosi. Data are means±SEM from three to four mice in each group.

FIGS. 6A-E are bar graphs of serum levels of cholesterol (mM) (FIG. 6A), triglyceride (mM) (FIG. 6B), free fatty acid (mM) (FIG. 6C), glucose (mg/dL) (FIG. 6D) and insulin (ng/ml) (Figure E) from fasting DIO mice treated with Rosi, NP-Rosi, iRGD-NP-Rosi and P3-NP-Rosi. Data are means±SEM from three to four mice in each group. FIG. 6F is a bar graph of insulin calculated as insulin level×FFA level. Data are means±SEM from three to four mice in each group.

FIG. 7 is an exemplary reaction scheme to synthesize peptide-NP constructs.

FIGS. 8A-D are bar graphs of relative expression levels in epididymal WAT from various treatment groups. Total RNAs were isolated from epididymal WAT from various groups treated with Rosi, NP-Rosi, iRGD-NP-Rosi and P3-NP-Rosi. Expression levels of UCP1 (FIG. 8A), CIDEA (FIG. 8B), DIO02 (FIG. 8C) and VEGFR2 (FIG. 8D) were quantified by qRT-PCR. Data are means±SEM from three to four mice in each group.

FIG. 9A is a bar graph of CD31⁺ area per field in inguinal WATs (×10³ μm²) from C57Bl/6 mice treated with PGE2, NP-PGE2, iRGD-NP-PGE2 and P3-NP-PGE2. Data are means±SEM from six samples in each group. FIG. 9B is a bar graph CD31 positive blood vessel area in epididymal WAT per optical field (×10³ μm²) from C57Bl/6 mice treated with PGE2, NP-PGE2, iRGD-NP-PGE2 and P3-NP-PGE2. Data are means±SEM from six samples in each group. FIG. 9C is a bar graph of relative expression level of Ucp1 in inguinal WAT from mice treated with PGE2, NP-PGE2, iRGD-NP-PGE2 and P3-NP-PGE2. Data are means±SEM from three to four mice in each group. FIG. 9E is a bar graph of relative expression level of Ucp1 in epididymal WAT from mice treated with PGE2, NP-PGE2, iRGD-NP-PGE2 and P3-NP-PGE2. Data are means±SEM from three to four mice in each group.

### DETAILED DESCRIPTION OF THE INVENTION

### I. Definitions

The terms “treating” or “preventing”, as used herein, can include preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The term “altered level of expression” of a marker, protein or gene refers to an expression level in a test sample (e.g., a sample derived from a subject during or following treatment for a metabolic disorder, such as diabetes and/or obesity), that is greater or less than the standard error of the assay employed to assess expression and may be at least two, three, four, five, six, seven, eight, nine, or ten times the expression level in a control sample (e.g., a sample from the subject prior to treatment), or the average expression level of the marker in several control samples.

The term “browning agent” refers to an agent that induces the conversion of WAT to BAT.

As used herein, the term “diabetes” refers to a number of well-known conditions. Insulin resistance is defined as a state in which circulating insulin levels in excess of the normal response to a glucose load are required to maintain the euglycemic state (Ford E S, et al. JAMA. (2002) 287:356-9). Insulin resistance, and the response of a subject with insulin resistance to therapy, may be quantified by assessing the homeostasis model assessment to insulin resistance (HOMA-IR) score, a reliable indicator of insulin resistance (Katsuki A, et al. Diabetes Care 2001; 24:362-5). The estimate of insulin resistance by the homeostasis assessment model (HOMA)-IR score is calculated with the formula (Galvin P, et al. Diabet Med 1992; 9:921-8): HOMA-IR=[fasting serum insulin (μU/mL)] times [fasting plasma glucose (mmol/L)/22.5]. Subjects with a predisposition for the development of impaired glucose tolerance (IGT) or type 2 diabetes are those having euglycemia with hyperinsulinemia are by definition, insulin resistant. A typical subject with insulin resistance is usually overweight or obese. The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749). Individuals likely to have insulin resistance are those who have two or more of the following attributes: 1) overweight or obese, 2) high blood pressure, 3) hyperlipidemia, 4) one or more 1st degree relative with a diagnosis of IGT or type 2 diabetes. Insulin resistance can be confirmed in these individuals by calculating HOMA-IR score. Insulin resistance may be defined as the clinical condition in which an individual has a HOMA-IR score >4.0 or a HOMA-IR score above the upper limit of normal as defined for the laboratory performing the glucose and insulin assays. Type 2 diabetes is defined as the condition in which a subject has a fasting blood glucose or serum glucose concentration greater than 125 mg/dl (6.94 mmol/L).

The terms “metabolic disorder” includes a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; and systemic responses in an organism, such as hormonal responses (e.g., insulin response).

In addition, metabolic disorders are associated with one or more discrete phenotypes. For example, body mass index (BMI) of a subject is defined as the weight in kilograms divided by the square of the height in meters, such that BMI has units of kg/m². In some embodiments, obesity is defined as the condition wherein the individual has a BMI equal to or greater than 30 kg/m². In another aspect, the term obesity is used to mean visceral obesity which can be defined in some embodiments as a waist-to-hip ratio of 1.0 in men and 0.8 in women, which, in another aspect defines the risk for insulin resistance and the development of pre-diabetes. In one embodiment, euglycemia is defined as the condition in which a subject has a fasting blood glucose concentration within the normal range, greater than 70 mg/dl (3.89 mmol/L) and less than 110 mg/dl (6.11 mmol/L). The word fasting has the usual meaning as a medical term. In one embodiment, impaired glucose tolerance (IGT), is defined as the condition in which a subject has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). The term impaired glucose tolerance is also intended to apply to the condition of impaired fasting glucose. In one embodiment, hyperinsulinemia is defined as the condition in which a subject with insulin resistance, with or without euglycemia, in which the fasting or postprandial serum or plasma insulin concentration is elevated above that of normal, lean individuals without insulin resistance, having a waist-to-hip ratio <1.0 (for men) or <0.8 (for women).

In some embodiments, “obesity” refers to a body mass index (BMI) of 30 kg/m² or more (National Institute of Health, Clinical Guidelines on the Identification, Evaluation, and Treatment of Overweight and Obesity in Adults (1998)). However, in some embodiments of the present invention, at least in part, is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, or 29.9 kg/m² or more, all of which are typically referred to as overweight (National Institute of Health, Clinical Guidelines on the Identification, Evaluation, and Treatment of Overweight and Obesity in Adults (1998)). The obesity may be due to any cause, whether genetic or environmental. In one embodiment, “prevention of obesity” refers to preventing obesity or an obesity-associated disorder from occurring if the treatment is administered prior to the onset of the obese condition. Moreover, if treatment is commenced in subjects already suffering from or having symptoms of obesity or an obesity-associated disorder, such treatment is expected to prevent, or to prevent the progression of obesity or the obesity-associated disorder.

The term “obesity-associated disorder” includes all disorders associated with or caused at least in part by obesity. Obesity-associated disorders include, for example, diabetes; cardiovascular disease; high blood pressure; deep vein thrombosis; osteoarthritis; obstructive sleep apnea; cancer and non-alcoholic fatty liver disease.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

“Topical administration”, as used herein, means the non-invasive administration to the skin, orifices, or mucosa. Topical administrations can be administered locally, i.e. they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endrotracheal administration. As used herein, the term “inhalation” refers to intake of air to the alveoli. The intake of air can occur through the mouth or nose.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The terms “sufficient” and “effective”, as used interchangeably herein, refer to an amount (e.g. mass, volume, dosage, concentration, and/or time period) needed to achieve one or more desired result(s).

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier”, as used herein, refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “hydrophilic”, as used herein, refers to substances that have strongly polar groups that readily interact with water.

The term “hydrophobic”, as used herein, refers to substances that lack an affinity for water, tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties.

The term “mean particle size”, as used herein, generally refers to the statistical mean particle size (diameter) of the particles in the composition. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering. Two populations can be said to have a “substantially equivalent mean particle size” when the statistical mean particle size of the first population of nanoparticles is within 20% of the statistical mean particle size of the second population of nanoparticles; more preferably within 15%, most preferably within 10%.

The terms “monodisperse” and “homogeneous size distribution”, as used interchangeably herein, describe a population of particles, microparticles, or nanoparticles all having the same or nearly the same size. As used herein, a monodisperse distribution refers to particle distributions in which 90% of the distribution lies within 5% of the mean particle size.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. The targeting moiety or a sufficient plurality of targeting moieties may be used to direct the localization of a particle or an active entity. The active entity may be useful for therapeutic, prophylactic, or diagnostic purposes.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene, T. W. and Wuts, P. G. M., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyryl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphεnylyl)-2-propy!oxycarbonyl, 2-bromobenzyloxycarbonyl, tert-butyl₇ tert-butyloxycarbonyl, 1-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

### II. Nanoparticles

The multimodal nanoparticle platform enables targeted drug delivery in a manner that produces a positive feedback loop. The nanoparticle contains binding moieties or targeting moieties that specifically bind to the target agent. A positive feedback loop is created when the nanoparticles release an inducing agent that causes a targeted cell, tissue or organ to increase the expression or bioavailability of a target agent specifically recognized by the nanoparticle. Representative targeting moieties include, but are not limited to, antibodies and antigen binding fragments thereof, aptamers, peptides, and small molecules. The binding moiety can be conjugated to a polymer that forms the nanoparticle. Typically the binding moiety is displayed on the outer shell of the nanoparticle. The outer shell serves as a shield to prevent the nanoparticles from being recognized by a subject's immune system thereby increasing the half-life of the nanoparticles in the subject. The nanoparticles also contain a hydrophobic core. In preferred embodiments, the hydrophobic core is made of a biodegradable polymeric material. The inner core carries therapeutic payloads and releases the therapeutic payloads at a sustained rate after systemic, intraperitoneal, oral, pulmonary, or topical administration. The nanoparticles also optionally include a detectable label, for example a fluorophore or NMR contrast agent that allows visualization of nanoparticles within plaques.

The nanoparticles may have any desired size for the intended use. The nanoparticles may have any diameter from 10 nm to 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In preferred embodiments the nanoparticles can have a diameter less than 400 nm, less than 300 nm, or less than 200 nm. The preferred range is between 50 nm and 300 nm.

Each of the components of the nanoparticles is discussed in more detail below.

A. Polymers

The nanoparticle can contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

The nanoparticle can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In preferred embodiments, the hydrophobic polymer is poly(lactic acid), poly(glycolic acid), or poly(lactic acid-co-glycolic acid).

The nanoparticle can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydrolyzable cross-linking groups to render the crosslinked polymer insoluble or sparingly soluble in water.

Biodegradable polymers in the nanoparticle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpyrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof.

The nanoparticles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In preferred embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both.

In preferred embodiments the nanoparticles contain a first amphiphilic polymer having a hydrophobic polymer block, a hydrophilic polymer block, and targeting moiety conjugated to the hydrophilic polymer block; and a second amphiphilic polymer having a hydrophobic polymer block and a hydrophilic polymer block but without the targeting moiety. The hydrophobic polymer block of the first amphiphilic polymer and the hydrophobic polymer block of the second amphiphilic polymer may be the same or different. Likewise, the hydrophilic polymer block of the first amphiphilic polymer and the hydrophilic polymer block of the second amphiphilic polymer may be the same or different.

In particularly preferred embodiments the nanoparticle contains biodegradable polyesters or polyanhydrides such as poly(lactic acid), poly(glycolic acid), and poly(lactic-co-glycolic acid). The nanoparticles can contain one more of the following polyesters: homopolymers including glycolic acid units, referred to herein as “PGA”, and lactic acid units, such as poly-L-lactic acid, poly-D-lactic acid, poly-DL-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The nanoparticles can also contain one or more polymer conjugates containing end-to-end linkages between the polymer and a targeting moiety or a detectable label. For example, a modified polymer can be a PLGA-PEG-peptide block polymer.

The nanoparticles can contain one or a mixture of two or more polymers. The nanoparticles may contain other entities such as stabilizers, surfactants, or lipids. The nanoparticles may contain a first polymer having a targeting moiety and a second polymer not having the targeting moiety. By adjusting the ratio of the targeted and non-targeted polymers, the density of the targeting moiety on the exterior of the particle can be adjusted. In some embodiments the ratio is optimized to enhance the targeting and/or adhesion of the nanoparticle to WAT or WAT vasculature.

The nanoparticles can contain an amphiphilic polymer having a hydrophobic end, a hydrophilic end, and a targeting moiety attached to the hydrophilic end. In some embodiments the amphiphilic macromolecule is a block copolymer having a hydrophobic polymer block, a hydrophilic polymer block covalently coupled to the hydrophobic polymer block, and a targeting moiety covalently coupled to the hydrophilic polymer block. For example, the amphiphilic polymer can have a conjugate having the structure A-B-X where A is a hydrophobic molecule or hydrophobic polymer, preferably a hydrophobic polymer, B is a hydrophilic molecule or hydrophilic polymer, preferably a hydrophilic polymer, and X is a targeting moiety. Preferred amphiphilic polymers include those where A is a hydrophobic biodegradable polymer, B is PEG, and X is a targeting moiety that targets, binds, and/or adheres to WAT or WAT vasculature.

In some embodiments the nanoparticle contains a first amphiphilic polymer having the structure A-B-X as described above and a second amphiphilic polymer having the structure A-B, where A and B in the second amphiphilic macromolecule are chosen independently from the A and B in the first amphiphilic macromolecule, although they may be the same.

One embodiment provides nanoparticles that are engineered to maximize half-life and targeting of the nanoparticles to WAT or WAT vasculature by adjusting the amount of PEG and the density of targeting moieties of the nanoparticles.

B. Targeting Moieties

The targeting moiety of the nanoparticle can be an antibody or antigen binding fragment thereof. The targeting moieties should have an affinity for a cell-surface receptor or cell-surface antigen on the target cells. The targeting moieties may result in internalization of the particle within the target cell.

The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

Additional targets that can be recognized by the targeting moiety include VEGF/KDR, Tie2, vascular cell adhesion molecule (VCAM), endoglin and α₅β₃ integrin/vitronectin. The targeting peptides can be covalently associated with the polymer of the outer shell and the covalent association can be mediated by a linker.

1. Peptide Targeting Moieties

In a preferred embodiment, the targeting moiety is a peptide. Specifically, the plaque targeted peptide can be, but is not limited to, one or more of the following: RGD, iRGD(CRGDK/RGPD/EC), LyP-1, P3(CKGCRAKDC), or their combinations at various molar ratios. The targeting peptides can be covalently associated with the polymer and the covalent association can be mediated by a linker. The peptides target to actively growing (angiogenic) vascular endothelial cells. Those angiogenic endothelial cells frequently appear in metabolic tissues such as adipose tissues.

2. Antibody Targeting Moieties

The targeting moiety can be an antibody or an antigen-binding fragment thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. The antigen binding portion of the antibody can be any portion that has at least one antigen binding site, such as Fab, F(ab′)₂, dsFv, sFv, diabodies, and triabodies. In certain embodiments, the antibody is a single chain antibody.

3. Aptamer Targeting Moieties

Aptamers are oligonucleotide or peptide sequences with the capacity to recognize virtually any class of target molecules with high affinity and specificity. Aptamers bind to targets such as small organics, peptides, proteins, cells, and tissues. Unlike antibodies, some aptamers exhibit stereoselectivity. The aptamers can be designed to bind to specific targets expressed on cells, tissues or organs.

C. Additional Moieties

The nanoparticles can contain one or more polymer conjugates containing end-to-end linkages between the polymer and a moiety. The moiety can be a targeting moiety, a detectable label, or a therapeutic, prophylactic, or diagnostic agent. For example, a polymer conjugate can be a PLGA-PEG-phosphonate. The additional targeting elements may refer to elements that bind to or otherwise localize the nanoparticles to a specific locale. The locale may be a tissue, a particular cell type, or a subcellular compartment. The targeting element of the nanoparticle can be an antibody or antigen binding fragment thereof, an aptamer, or a small molecule (less than 500 Daltons). The additional targeting elements may have an affinity for a cell-surface receptor or cell-surface antigen on a target cell and result in internalization of the particle within the target cell.

The nanoparticles can also contain a detectable label, such as, a radioisotope, a fluorophore (e.g., fluorescein isothiocyanate (FITC), phycoerythrin (PE)), an enzyme (e.g., alkaline phosphatase, horseradish peroxidase), element particles (e.g., gold particles) or a contrast agent. These may be encapsulated within, dispersed within, or conjugated to the polymer.

For example, a fluorescent label can be chemically conjugated to a polymer of the nanoparticle to yield a fluorescently labeled polymer. In other embodiments the label is a contrast agent. A contrast agent refers to a substance used to enhance the contrast of structures or fluids within the body in medical imaging. Contrast agents are known in the art and include, but are not limited to agents that work based on X-ray attenuation and magnetic resonance signal enhancement. Suitable contrast agents include iodine and barium.

D. Inner Core

The inner core of the nanoparticle is hydrophobic and can be loaded with a therapeutic agent. In some embodiments, the therapeutic agent is a target inducing agent.

1. Target Inducing Agent

The inner core of the nanoparticle can contain a target inducing agent. The target inducing agent induces the expression or bioavailability of the target recognized by the targeting moiety of the nanoparticle. The inducing agent can be growth factors such as: adrenomedullin, angiopoietin, autocrine motility factor, bone morphogenetic proteins, brain-derived neurotrophic factor, epidermal growth factor, erythropoietin, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin-like growth factor, migration-stimulating factor, myostatin, nerve growth factor, neurotrophins, platelet-derived growth factor, thrombopoietin, transforming growth factor alpha, transforming growth factor beta, tumor necrosis factor-alpha, and vascular endothelial growth factor. It will be appreciated that when nanoparticles are loaded with one or more these or other growth factors, the nanoparticle targeting moiety will recognize and selectively bind to a target whose expression is induced by the growth factor.

In a preferred embodiment, the inducing agent induces the conversion of WAT to BAT. These agents are referred to as “browning agents”. Representative browning agents include, but are not limited to β-adrenergic agonists, leptin, TLQP-21. brain-derived neurothrophic factor, prostaglandins, cardiac natriuretic peptides, PPARγ ligands, PPARα ligands, retinoids, thyroid hormones, AMPK activators, Irisin, fibroblast growth factor 21, and bone morphogenetic protein (M. L. Bonet et al., Biochimica et Biophysica Acta 1831, 969-985(2013)). Preferred browning agents include PPARγ activators (e.g. Rosiglitazone, (RS)-5-[4-(2-[methyl(pyridin-2-yl)amino]ethoxy)benzyl]thiazolidine-2,4-dione, Pioglitazone, (RS)-5-(4-[2-(5-ethylpyridin-2-yl)ethoxy]benzyl)thiazolidine-2,4-dione, Troglitazone, (RS)-5-(4-[(6-hydroxy-2,5,7,8-tetramethylchroman-2-yl)methoxy]benzyl)thiazolidine-2,4-dione etc.), prostagladin E2 analog (PGE2, (5Z,11α,13E,15S)-7-[3-hydroxy-2-(3-hydroxyoct-1-enyl)-5-oxo-cyclopentyl] hept-5-enoic acid etc.), beta3 adrenoceptor agonist (CL 316243, Disodium 5-[(2R)-2-[[(2R)-2-(3-Chlorophenyl)-2-hydroxyethyl]amino]propyl]-1,3-benzodioxole-2,2-dicarboxylate hydrate, etc.), Fibroblast Growth Factor 21 (FGF-21), and Irisin.

2. Therapeutic Agent

The inner core can be loaded with a therapeutic, prophylactic or diagnostic agent. These can be proteins, peptides, amino acids, nucleic acids, carbohydrates, lipids, small molecules, or combinations thereof. In certain embodiments, the inducing agent and the therapeutic agent are the same. In other embodiments the inducing agent and the therapeutic agents are different. Exemplary therapeutic agents include, but are not limited to, antibiotics, anti-inflammatory agents, chemotherapeutic agents, analgesics, hormones, steroids, cytotoxic agents, growth factors, cytokines, and combinations thereof.

In other embodiments, the nanoparticles can be loaded with oligonucleotides such as iRNA, RNA, DNA, and siRNA. The oligonucleotides can be single or double stranded. The oligonucleotides can be designed to inhibit or reduce the expression of targeted genes. In one embodiment, the oligonucleotides are designed to inhibit genes in adipocytes and thereby assist in transforming white adipocytes into brown adipocyte-like cells.

Therapeutics include angiogenesis stimulators and inhibitors, proliferation stimulators and inhibitors, and agents modulating adipocyte metabolism.

The angiogenesis factors are divided into three classes.

The first class consists of the VEGF family and the angiopoietins. These act especially on the endothelial cells. VEGF belongs to the VEGF family. There are six members in this family. These include VEGF-A (or VEGF), PlGF, VEGF-B, VEGF-C, VEGF-D, and orf virus VEGF (VEGF-E). VEGF is vital for survival and development of the fetus. VEGF works by stimulating the breakdown of the ECM along with multiplication, movement and tube formation of endothelial cells. It helps these cells express uPA, PAI-1, uPAR, and MMP-1.

The second class contains direct-acting molecules like cytokines, chemokines and angiogenic enzymes. These target other cells as well. One of the members of this group is the FGF-2 that was one of the first angiogenic peptides to be characterized. The FGF family has 19 members. FGF 2 is vital for angiogenesis. It induces multiplication and movement of the cells as well as uPA production by endothelial cells. FGF-2 induces tube formation in collagen gels and alters integrin expression that helps in angiogenesis. FGF21 loaded nanoparticles can help regulate adipose tissue transformation and angiogenesis.

The third group of angiogenic molecules are indirect-acting factors that act by release of direct-acting factors from macrophages, endothelial or tumor cells rather than directly on the endothelial cells. Agents of this group include tumor necrosis factors (TNF a and b).

The process of adipocyte differentiation from a precursor preadipocyte to a fully mature adipocyte follows a precisely ordered and temporally regulated series of events. Adipocyte precursor cells emerge from mesenchymal stem cells (MSCs) that are themselves derived from the mesodermal layer of the embryo. The pluripotent MSCs receive extracellular cues that lead to the commitment to the preadipocyte lineage. Preadipocytes cannot be morphologically distinguished from their precursor MSCs but they have lost the ability to differentiate into other cell types. This initial step in adipocyte differentiation is referred to as determination and leads to proliferating preadipocytes undergoing a growth arrest. This initial growth arrest occurs coincident with the expression of two key transcription factors, CCAAT/enhancer binding protein alpha (C/EBPα) and peroxisome proliferator-activated receptor gamma (PPARγ). Following the induction of these two critical transcription factors there is a permanent period of growth arrest followed by expression of the fully differentiated adipocyte phenotype. This latter phase of adipogenesis is referred to as terminal differentiation.

Although PPARγ and C/EBPα are the most important factors regulating adipogenesis additional transcription factors are known to influence this process. These additional factors include sterol-regulated element binding protein 1c (SREBP1c, also known as ADD1 for adipocyte differentiation-1), signal transducers and activators of transcription 5 (STAT5), AP-1 and members of the Krüppel-like factor (KLF4, KLF5, and KLF15) family as well as C/EBP beta (β) and C/EBP delta (δ). Although these numerous transcription factors have been shown to influence overall adipogenesis, either positively or negatively, PPARγ is the only one that is necessary for adipogenesis to take place. In fact, in the absence of PPARγ, adipocyte differentiation fails to occur and as yet no factor has been identified that can rescue adipogenesis in the absence of PPARγ. In spite of this fact, expression of PPARγ does not commence during the initial activation of adipocyte differentiation but only after the responses elicited by STAT5, KLF4, KLF5, AP-1, SREBP1c, and C/EBPβ and C/EBPδ are exerted.

PPARγ was originally identified as being expressed in differentiating adipocytes and as indicated above it is now recognized as a master regulator of adipogenesis. PPARγ was identified as the target of the thiazolidinedione (TZD) class of insulin-sensitizing drugs. The mechanism of action of the TZDs is a function of the activation of PPARγ and the consequent induction of genes necessary for differentiation of adipocytes. The human PPARγ gene (symbol PPARG) is located on chromosome 3p25 spanning over 100 kb and composed of 9 exons encoding two biologically active isoforms as a consequence of alternative mRNAs and translational start codon usage. The principal protein products of the PPARG gene are identified as PPARγ1 and PPARγ2. PPARγ1 is encoded for by exons A1 and A2 then common exons 1 through 6. PPARγ2 is encoded by exon B and common exons 1 through 6. PPARγ2 is almost exclusively expressed in adipocytes. Like all nuclear receptors the PPARγ proteins contain a DBD and a LBD. In addition, like PPARα, the PPARγ proteins contain a ligand-dependent activation function domain (identified as AF-2) and a ligand-independent activation function domain (identified as AF-1). The AF-2 domain resides in the LBD and the AF-1 domain is in the N-terminal region of the PPARγ proteins. PPARγ2 protein contains an additional 30 N-terminal amino acids relative to PPARγ1 and these additional amino acids confer a 5-6-fold increase in the transcription-stimulating activity of AF-1 when compared to the same domain in the PPARγ1 protein. Expression of PPARγ1 is nearly ubiquitous. PPARγ2 is expressed near exclusively in white adipose tissue (WAT) where it is involved in lipid storage and in BAT where it is involved in energy dissipation.

During adipocyte differentiation several upstream genes are required for the activation of the PPARG gene. These include C/EBPβ and C/EBPδ, SREBP-1c, KLF5, KLF15, zinc-finger protein 423 (Zfp423), and early B-cell factor (Ebf1). In the process of adipocyte differentiation PPARγ activates nearly all of the genes required for this process. These genes include aP2 which is required for transport of free fatty acids (FFAs) and perilipin which is a protein covering the surface of mature lipid droplets in adipocytes. Additional genes regulated by PPARγ that are involved in lipid metabolism or glucose homeostasis include lipoprotein lipase (LPL), acyl-CoA synthase (ACS), acetyl-CoA acetyltransferase 1 (ACAT1), several phospholipase A (PLA) genes, adiponectin, the gluconeogenic enzyme PEPCK, and glycerol-3-phosphate dehydrogenase (GPD1). PPARγ also functions in macrophage lipid metabolism by inducing the expression of the macrophage scavenger receptor, CD36. The CD36 receptor is also known as fatty acid translocase (FAT) and it is one of the receptors responsible for the cellular uptake of fatty acids.

The role of SREBP-1c in activation of adipocyte differentiation is thought to be the result of this transcription factor initiating the expression of genes that, as part of their activities, generate PPARγ ligands. This fact explains the necessity for SREBP expression to precede that of PPARγ. In spite of this fact it has been shown that mice lacking SREBP-1 do not display significant reductions in the amount of WAT. However, levels of SREBP-2 are increased in these animals indicating that this may be a compensatory mechanism. Although loss of SREBP-1 expression does not result in a significant deficit in adipose tissue development, ectopic overexpression of SREBP-1c does enhance the adipogenic activity of PPARγ.

The C/EBP family of transcription factors were among the first to be shown to play a role in overall adipocyte differentiation. The three members of the family (C/EBPα, C/EBPβ and C/EBPδ) are highly conserved basic-leucine zipper containing transcription factors. The importance of these factors in adipogenesis has been demonstrated in knockout mouse models. For example, whole body disruption of C/EBPα expression results in death shortly after birth due to liver defects, hypoglycemia, and failure of WAT or BAT accumulation. Using knockout mice it has been determined that the roles of C/EBPβ and C/EBPδ are exerted early in the process of adipocyte differentiation whereas those of C/EBPα are required later. In fact, expression of C/EBPα is induced late in adipogenesis and is most abundant in mature adipocytes. The expression of both C/EBPα and PPARγ is, in part, regulated by the actions of C/EBPβ and C/EBPδ. One of the major effects of the expression of C/EBPα in adipocytes is enhanced insulin sensitivity of adipose tissue. This later fact is demonstrated by the fact that C/EBPα knockout does not abolish adipogenesis but the WAT is not sensitive to the actions of insulin.

The general model of transcription factor activation of adipogenesis indicates that AP-1, STAT5, KLF4, and KLF5 are activated early and result in the transactivation of C/EBPβ and C/EBPδ. These latter two factors in turn activate the expression of SREPB-1 and KLF15 which leads to the activation of PPARγ and C/EBPα. It is important to keep in perspective that it is not only transcription factor activation of adipocyte precursors that controls adipogenesis. There is also a balance exerted at the level of transcription factor-mediated inhibition of adipogenesis. Some of the factors that are anti-adipogeneic include members of the Krüppel-like factor family, KLF2 and KLF3. GATA2 and GATA3 also exert anti-adipogenic activity. GATA factors are so-called because they bind DNA elements that contain a core GATA sequence. Two of the interferon regulatory factor family of transcription factors, IRF3 and IRF4, oppose the process of adipogenesis as well.

These factors can be induced or inhibited through the use of oligonucleotide molecules such as siRNA and microRNA binding agents.

In the U.S., there are currently thirteen approved anti-cancer therapies with recognized antiangiogenic properties in oncology. These agents, which interrupt critical cell signaling pathways involved in tumor angiogenesis and growth, comprise three primary categories: 1) monoclonal antibodies directed against specific proangiogenic growth factors and/or their receptors; and 2) small molecule tyrosine kinase inhibitors (TKIs) of multiple proangiogenic growth factor receptors; 3) inhibitors of mTOR (mammalian target of rapamycin). In addition, at least two other approved angiogenic agents may indirectly inhibit angiogenesis through mechanisms that are not completely understood. Finally, in the field of dermatology, there are several agents used for neoplasms of the skin. In the U.S., there are currently thirteen approved anti-cancer therapies with recognized antiangiogenic properties in oncology. These agents, which interrupt critical cell signaling pathways involved in tumor angiogenesis and growth, comprise three primary categories: 1) monoclonal antibodies directed against specific proangiogenic growth factors and/or their receptors; and 2) small molecule tyrosine kinase inhibitors (TKIs) of multiple proangiogenic growth factor receptors; 3) inhibitors of mTOR (mammalian target of rapamycin). In addition, at least two other approved angiogenic agents may indirectly inhibit angiogenesis through mechanisms that are not completely understood. Finally, in the field of dermatology, there are several agents used for neoplasms of the skin.

Two mTOR inhibitors, temsirolimus (Torisel) and everolimus (Afinitor), are currently approved as anti-cancer therapy.

Presently approved anti-angiogenic therapies for ophthalmic conditions are biologic agents that inhibit VEGF. There are currently three approved antiangiogenic therapies for ophthalmic diseases: an anti-VEGF aptamer (pegaptanib, Macugen); a Fab fragment of a monoclonal antibody directed against VEGF-A (ranibizumab, Lucentis); and a fusion protein that binds to VEGF-A, VEGF-B, and PlGF (afilbercept, Eylea).

Still other embodiments provide nanoparticles loaded with two or more therapeutic agents described above.

### III. Methods of Making Nanoparticles

A. Polymer Conjugates

Methods of polymer synthesis are described, for instance, in Braun et al. (2005) Polymer Synthesis: Theory and Practice. New York, N.Y.: Springer. The polymers may be synthesized via step-growth polymerization, chain-growth polymerization, or plasma polymerization.

In some embodiments an amphiphilic polymer is synthesized starting from a hydrophobic polymer terminated with a first reactive coupling group and a hydrophilic polymer terminated with a second reactive coupling group capable of reacting with the first reactive coupling group to form a covalent bond. One of either the first reactive coupling group or the second reactive coupling group can be a primary amine, where the other reactive coupling group can be an amine-reactive linking group such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. One of either the first reactive coupling group or the second reactive coupling group can be an aldehyde, where the other reactive coupling group can be an aldehyde reactive linking group such as hydrazides, alkoxyamines, and primary amines. One of either the first reactive coupling group or the second reactive coupling group can be a thiol, where the other reactive coupling group can be a sulfhydryl reactive group such as maleimides, haloacetyls, and pyridyl disulfides.

In preferred embodiments a hydrophobic polymer terminated with an amine or an amine-reactive linking group is coupled to a hydrophilic polymer terminated with complimentary reactive linking group. For example, an NHS ester activated PLGA can be formed by reacting PLGA-CO(OH) with NHS and a coupling reagent such as dicyclohexylcarbodiimide (DCC) or ethyl(dimethylaminopropyl) carbodiimide (EDC). The NHS ester activated PLGA can be reacted with a hydrophilic polymer terminated with a primary amine, such as a PEG-NH₂ to form an amphiphilic PLGA-b-PEG block copolymer.

In some embodiments a conjugate of an amphiphilic polymer with a targeting moiety is formed using the same or similar coupling reactions. In some embodiments the conjugate is made starting from a hydrophilic polymer terminated on one end with a first reactive coupling group and terminated on a second end with a protective group. The hydrophilic polymer is reacted with a targeting moiety having a reactive group that is complimentary to the first reactive group to form a covalent bond between the hydrophilic polymer and the targeting moiety. The protective group can then be removed to provide a second reactive coupling group, for example to allow coupling of a hydrophobic polymer block to the conjugate of the hydrophilic polymer with the targeting moiety. A hydrophobic polymer terminated with a reactive coupling group complimentary to the second reactive coupling group can then be covalently coupled to form the conjugate. Of course, the steps could also be performed in reverse order, i.e. a conjugate of a hydrophobic polymer and a hydrophilic polymer could be formed first followed by deprotection and coupling of the targeting moiety to the hydrophilic polymer block.

In some embodiments a conjugate is formed having a moiety conjugated to both ends of the amphiphilic polymer. For example, an amphiphilic polymer having a hydrophobic polymer block and a hydrophilic polymer block may have targeting moiety conjugated to the hydrophilic polymer block and an additional moiety conjugated to the hydrophobic polymer block. In some embodiments the additional moiety can be a detectable label. In some embodiments the additional moiety is a therapeutic, prophylactic, or diagnostic agent. For example, the additional moiety could be a moiety used for radiotherapy. The conjugate can be prepared starting from a hydrophobic polymer having on one end a first reactive coupling group and a another end first protective group and a hydrophilic polymer having on one end a second reactive coupling group and on another end a second protective group. The hydrophobic polymer can be reacted with the additional moiety having a reactive coupling group complimentary to the first reactive coupling group, thereby forming a conjugate of the hydrophobic polymer to the additional moiety. The hydrophilic polymer can be reacted with a targeting moiety having a reactive coupling group complimentary to the second reactive coupling group, thereby forming a conjugate of the hydrophilic polymer to the targeting moiety. The first protective group and the second protective group can be removed to yield a pair of complimentary reactive coupling groups that can be reacted to covalently link the hydrophobic polymer block to the hydrophilic polymer block.

B. Emulsion Methods

In some embodiments, a multimodal nanoparticle is prepared using an emulsion solvent evaporation method. For example, a polymeric material is dissolved in a water immiscible organic solvent and mixed with a drug solution or a combination of drug solutions. In some embodiments a solution of a therapeutic, prophylactic, or diagnostic agent to be encapsulated is mixed with the polymer solution. The polymer can be, but is not limited to, one or more of the following: PLA, PGA, PCL, their copolymers, polyacrylates, the aforementioned PEGylated polymers, the aforementioned Polymer-drug conjugates, the aforementioned polymer-peptide conjugates, or the aforementioned fluorescently labeled polymers, or various forms of their combinations. The drug molecules can be, but are not limited to, one or a more of the following: PPARgamma activators (e.g. Rosiglitazone, (RS)-5-[4-(2-[methyl(pyridin-2-yl)amino]ethoxy)benzyl]thiazolidine-2,4-dione, Pioglitazone, (RS)-5-(4-[2-(5-ethylpyridin-2-yl)ethoxy]benzyl)thiazolidine-2,4-dione, Troglitazone, (RS)-5-(4-[(6-hydroxy-2,5,7,8-tetramethylchroman-2-yl)methoxy]benzyl)thiazolidine-2,4-dione etc.), prostagladin E2 analog (PGE2, (5Z,11α,13E,15S)-7-[3-hydroxy-2-(3-hydroxyoct-1-enyl)-5-oxo-cyclopentyl] hept-5-enoic acid etc.), beta3 adrenoceptor agonist (CL 316243, Disodium 5-[(2R)-2-[[(2R)-2-(3-Chlorophenyl)-2-hydroxyethyl]amino]propyl]-1,3-benzodioxole-2,2-dicarboxylate hydrate, etc.), Fibroblast Growth Factor 21 (FGF-21), Irisin, RNA, DNA, chemotherapeutic compounds, nuclear magnetic resonance (NMR) contrast agents, or combinations thereof. The water immiscible organic solvent, can be, but is not limited to, one or more of the following: chloroform, dichloromethane, and acyl acetate. The drug can be dissolved in, but is not limited to, one or more of the following: acetone, ethanol, methanol, isopropyl alcohol, acetonitrile and Dimethyl sulfoxide (DMSO).

In some embodiments the polymer solution contains one or more polymer conjugates as described above. The polymer solution can contain a first amphiphilic polymer conjugate having a hydrophobic polymer block, a hydrophilic polymer block, and a targeting moiety conjugated to the hydrophilic end. In preferred embodiments the polymer solution contains one or more additional polymers or amphiphilic polymer conjugates. For example the polymer solution may contain, in addition to the first amphiphilic polymer conjugate, one or more hydrophobic polymers, hydrophilic polymers, lipids, amphiphilic polymers, polymer-drug conjugates, or conjugates containing other targeting moieties. By controlling the ratio of the first amphiphilic polymer to the additional polymers or amphiphilic polymer conjugates, the density of the targeting moieties can be controlled. The first amphiphilic polymer may be present from 1% to 100% by weight of the polymers in the polymer solution. For example, the first amphiphilic polymer can be present at 10%, 20%, 30%, 40%, 50%, or 60% by weight of the polymers in the polymer solution.

An aqueous solution is then added into the resulting mixture solution to yield emulsion solution by emulsification. The emulsification technique can be, but not limited to, probe sonication or homogenization through a homogenizer. The plaque-targeted peptides or fluorophores or drugs may be associated with the surface of; encapsulated within, surrounded by, and/or distributed throughout the polymeric matrix of this inventive particle.

C. Nanoprecipitation Method

In another embodiment, a multimodal nanoparticle is prepared using nanoprecipitation methods or microfluidic devices. A polymeric material is mixed with a drug or drug combinations in a water miscible organic solvent. The polymer can be, but is not limited to, one or more of the following: PLA, PGA, PCL, their copolymers, polyacrylates, the aforementioned PEGylated polymers, the aforementioned Polymer-drug conjugates, the aforementioned polymer-peptide conjugates, or the aforementioned fluorescently labeled polymers, or various forms of their combinations. The drug molecules can be, but are not limited to, one or more of the following: PPARgamma activators (e.g. Rosiglitazone, (RS)-5-[4-(2-[methyl(pyridin-2-yl)amino]ethoxy)benzyl]thiazolidine-2,4-dione, Pioglitazone, (RS)-5-(4-[2-(5-ethylpyridin-2-yl)ethoxy]benzyl)thiazolidine-2,4-dione, Troglitazone, (RS)-5-(4-[(6-hydroxy-2,5,7,8-tetramethylchroman-2-yl)methoxy]benzyl)thiazolidine-2,4-dione etc.), prostagladin E2 analog (PGE2, (5Z,11α,13E,15S)-7-[3-hydroxy-2-(3-hydroxyoct-1-enyl)-5-oxo-cyclopentyl] hept-5-enoic acid etc.), beta3 adrenoceptor agonist (CL 316243, Disodium 5-[(2R)-2-[[(2R)-2-(3-Chlorophenyl)-2-hydroxyethyl]amino]propyl]-1,3-benzodioxole-2,2-dicarboxylate hydrate, etc.), RNA, DNA, chemotherapeutic compounds, nuclear magnetic resonance (NMR) contrast agents, or combinations thereof. The water miscible organic solvent, can be, but is not limited to, one or more of the following: acetone, ethanol, methanol, isopropyl alcohol, acetonitrile and Dimethyl sulfoxide (DMSO). The resulting mixture solution is then added to a polymer non-solvent, such as an aqueous solution, to yield nanoparticle solution. The plaque-targeted peptides or fluorophores or drugs may be associated with the surface of, encapsulated within, surrounded by, and/or distributed throughout the polymeric matrix of this inventive particle.

D. Microfluidics

Methods of making nanoparticles using microfluidics are known in the art. Suitable methods include those described in U.S. Patent Application Publication No. 2010/0022680 A1 by Karnik et al. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the nanoparticles having the desired size and density of moieties on the surface. By varying the pressure and flow rate in the inlet channels and the nature and composition of the fluid sources nanoparticles can be produced having reproducible size and structure.

### IV. Formulations

The formulations contain an effective amount of nanoparticles in a pharmaceutical carrier appropriate for administration to an individual in need thereof to treat one or more symptoms of a disease or disorder. The formulations can be administered parenterally (e.g., by injection or infusion), topically (e.g., to a mucosal surface such as the mouth, lungs, intranasal, intravaginally, etc.), or enterally.

A. Parenteral Formulations

The nanoparticles can be formulated for parenteral delivery, such as injection or infusion, in the form of a solution or suspension, or a powder. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the nanoparticles can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles.

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the nanoparticles in the required amount in the appropriate solvent or dispersion medium with one or more of the excipients listed above, as required, followed by filtered sterilization. Generally, dispersions are prepared by incorporating the various sterilized nanoparticles into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the nanoparticle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration are preferably in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

B. Topical and Mucosal Formulations

The nanoparticles can be formulated for topical administration. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof.

In some embodiments, the nanoparticles can be administered as a liquid formulation, such as a solution or suspension, a semi-solid formulation, such as a lotion or ointment, or a solid formulation. In some embodiments, the nanoparticles are formulated as liquids, including solutions and suspensions, such as eye drops or as a semi-solid formulation, such as ointment or lotion for topical application to the skin, to the mucosa, such as the eye or vaginally or rectally.

The formulation may contain one or more excipients, such as emollients, surfactants, emulsifiers, and penetration enhancers. “Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

An “oil” is a composition containing at least 95% wt of a lipophilic substance. Examples of lipophilic substances include, but are not limited to, naturally occurring and synthetic oils, fats, fatty acids, lecithins, triglycerides and combinations thereof.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove. The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of the nanoparticles in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant may consist primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more nanoparticles to a patient in need thereof. For topical applications, repeated application can be done or a patch can be used to provide continuous administration of the noscapine analogs over an extended period of time.

C. Enteral Formulations

The drug-encapsulated polymeric NPs can be further surface modified with the Fc portion of IgG. Fc receptor (FcRn) present on the apical membrane of absorptive epithelial cells predominately in the duodenum section of the small intestine. The FcRn is responsible for active transport of IgG antibodies across the intestinal epithelium through the process of transcytosis. Using the Fc portion of IgG to target drug-encapsulated polymeric NPs to the FcRn will allow drug-loaded NPs to be actively transported across the intestinal epithelium and enter systemic circulation after oral administration.

The nanoparticles can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition which may include plasticizers, pigments, colorants, stabilizing agents, and glidants. Formulations can be prepared using one or more pharmaceutically acceptable excipients, including diluents, preservatives, binders, lubricants, disintegrators, swelling agents, fillers, stabilizers, and combinations thereof. Delayed release dosage formulations can be prepared as described in standard references such as “Pharmaceutical dosage form tablets”, eds. Liberman, et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The nanoparticles may be coated, for example to delay release once the particles have passed through the acidic environment of the stomach. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers and/or pH dependent polymers, with or without water insoluble/water soluble non polymeric excipient, to produce the desired release profile. The coating is either performed on dosage form (matrix or simple) which includes, but not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as crosslinked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

### V. Methods of Use

The nanoparticles, nanoparticle formulations, and pharmaceutical compositions containing the nanoparticles can be used to treat diseases and disorders by targeted delivery of therapeutic agents to specific cells, tissues and organs. Diseases that can be treated include, but are not limited to metabolic diseases and disorders, such as obesity and diabetes.

Metabolic diseases include obesity, hyperlipidemia and insulin resistance or a disease associated with a lack of mitochondria, e.g., diabetes, neurodegeneration, and aging.

In some embodiments the metabolic disorder is obesity, insulin resistance, hyperinsulinemia, hypoinsulinemia, type II diabetes, hypertension, hyperhepatosteatosis, hyperuricemia, fatty liver, non-alcoholic fatty liver disease, polycystic ovarian syndrome, acanthosis nigricans, hyperphagia, endocrine abnormalities, triglyceride storage disease, Bardet-Biedl syndrome, Lawrence-Moon syndrome, Prader-Labhart-Willi syndrome, or muscle hypoplasia.

Treatment of the obesity facilitates treat of obesity-associated disorders such as diabetes; cardiovascular disease; high blood pressure; deep vein thrombosis; osteoarthritis; obstructive sleep apnea; cancer and non-alcoholic fatty liver disease.

A. Methods of Treating Metabolic Disorders

The methods and compositions are useful for the treatment of diseases, including metabolic diseases and weight-related disorders. To treat obesity, the methods include inducing WAT in a subject to change into BAT or brown-like adipose tissue.

In some embodiments, the methods include identifying a subject in need of treatment (e.g., an overweight or obese subject, e.g., with a body mass index (BMI) of 25-29 or 30 or above or a subject with a weight related disorder) and administering to the subject an effective amount of the nanoparticles loaded with a browning agent and targeted to WAT or WAT vasculature. A subject in need of treatment can be selected based on the subject's body weight or body mass index. In some embodiments, the methods include evaluating the subject for one or more of weight, adipose tissue stores, adipose tissue morphology, insulin levels, insulin metabolism, glucose levels, thermogenic capacity, and cold sensitivity. In some embodiments, subject selection can include assessing the amount or activity of brown adipose tissue in the subject and recording these observations.

B. Differentiation of WAT into Brown Adipose-Like Cells

Methods and compositions are disclosed to increase BAT activity or energy expenditure by increasing the total amount of BAT in a subject. This can be achieved through multiple mechanisms, such as differentiation of WAT into brown adipose-like cells and differentiation of stem/progenitor cells to brown adipose cells, e.g. inducing differentiation of artery-derived cells into brown adipose-like cells.

Adipocytes are central to the control of energy balance and lipid homeostasis. The ability to store excess energy in adipose tissue is an important evolutionary adaptation. There are two types of fat or adipose tissue: white adipose tissue (WAT), the primary site of energy storage, and brown adipose tissue (BAT), specialized for energy expenditure and thermogenesis. An inverse correlation exists between the amount of brown adipose tissue and body mass index, with obese individuals having significantly less of the tissue than lean individuals; this suggests that brown fat may be an important factor in maintaining a lean phenotype or that the obese phenotype has led to the diminution in size and/or activity of the BAT depots.

Adipose tissue is composed, in part, of adipocytes or adipose cells specific for WAT or BAT. Adipocytes can also produce adipokines, such as tumor necrosis factor α (TNFα), leptin, resistin, retinol binding protein 4 (RBP4), apelin, and adiponectin, to modulate systemic metabolism. The inability to properly store triglycerides in adipose tissue results in adverse effects on glucose metabolism in the liver and skeletal muscle. In contrast with WAT, the physiological role of BAT is to metabolize fatty acids and expend energy through thermogenesis. This specialized activation function of brown adipose cells derives from high mitochondrial content and the ability to uncouple cellular respiration through physical or chemical stimulation or signaling through upstream receptors of uncoupling protein (UCP) to generate heat. Thermogenesis is the heat production caused by the metabolic rate activated by exposure to cold. For example, brown adipose cells become activated and exhibit thermogenic potential due to proton leak across the mitochondrial membrane that generates heat. This functional potential can also be stimulated by exposure to at least one of a catecholamine, like norepinephrine, cyclic AMP and leptin. Due to these functional differences between WAT and BAT, the ratio of WAT to BAT can affect systemic energy balance that may contribute to the development of obesity.

Augmenting the number of BAT cells to increase overall energy expenditure in a subject can provide a mechanism to treat metabolic disorders, such as obesity, diabetes and hyperlipidemia. In one embodiment, brown adipose tissue can be augmented by inducing WAT into brown adipose-like cells or the differentiation of the adipocyte precursor cells into brown adipose-like cells.

Inducing differentiation of WAT or adipocyte precursor cells to brown adipose-like cells can be performed by treatment of adipocytes with compounds such as ligands for peroxisome proliferator-activated receptor γ (PPARγ, pioglitazone, rosiglitazone, AVANDIA™) as described above. In one embodiment, WAT is induced to form brown adipose-like cells by administering an effective amount of the nanoparticles loaded with a browning agent.

Adipocyte differentiation may be detected through expression of one or more adipose related markers. As used herein, the term “adipose related marker” includes adipocyte markers, brown adipocyte markers and brown adipose-like markers. The adipose related marker, such as adipocyte markers, may be elevated to a higher level as compared to untreated adipocyte precursor cells. The adipose related marker may be an adipocyte marker or a brown adipocyte marker. Examples of adipocyte markers can include, but are not limited to, fatty acid binding protein 4 (aP2), peroxisome proliferator activated receptor α (PPARα) peroxisome proliferator activated receptor γ (PPARγ), adiponectin (AND or ADIPOQ), uncoupling protein 1 (UCP-1), PR domain containing protein 16 (PRDM16), PPAR coactivator-1α (PGC-1α), CCAAT/enhancer binding protein .beta. (C/EBPβ), cell death-inducing DFFA-like effector A (CIDE-A), and elongation of very long chain fatty acids like protein 3 (ELOVL3). Examples of brown adipocyte markers can include, but are not limited to, uncoupling protein 1 (UCP-1), PR domain containing protein 16 (PRDM16), PPAR coactivator-1α (PGC-1α), CCAAT/enhancer binding protein .beta. (C/EBPβ), cell death-inducing DFFA-like effector A (CIDE-A), and elongation of very long chain fatty acids like protein 3 (ELOVL3).

C. Obesity

The nanoparticles can specifically deliver therapeutic compounds or imaging agents to white fat tissue to treat obesity. Massive expansion of adipose tissues such as white fat tissue (WAT) leads to obesity, which has become a major threat to human health throughout the world. Current obesity therapeutic approaches include restriction of food intake, enhanced exercise, medication and plastic surgery. Limited therapeutic agents are available for treating obesity in clinic due to a complex interplay among genetic, environmental and cultural factors. In addition, numerous weight-loss drugs have been abandoned because of undesired side effects. One of the top reasons is the drugs have broad targeting spectrums that affect multiple organs and tissues.

Thus, one embodiment provides a method for treating obesity or an obesity-related disorder in subject in need thereof by administering nanoparticles containing a hydrophobic copolymer core that contains an adipose tissue browning agent in an amount effective to increase vascularization and adipose tissue transformation and a hydrophilic polymer corona surrounding the hydrophobic copolymer core, wherein the hydrophilic polymer corona includes a vasculature targeting moiety that targets the nanoparticle to adipose tissue angiogenic vessels to facilitate homing of the nanoparticle to white adipose tissue and increase angiogenesis in the white adipose tissue thereby amplifying delivery of additional nanoparticles to the white adipose tissue. In certain embodiments, the biodegradable nanoparticle self-assembles to produce a nanoparticle loaded with the adipose tissue browning agent.

D. Methods for Reducing Serum Cholesterol and Triglycerides

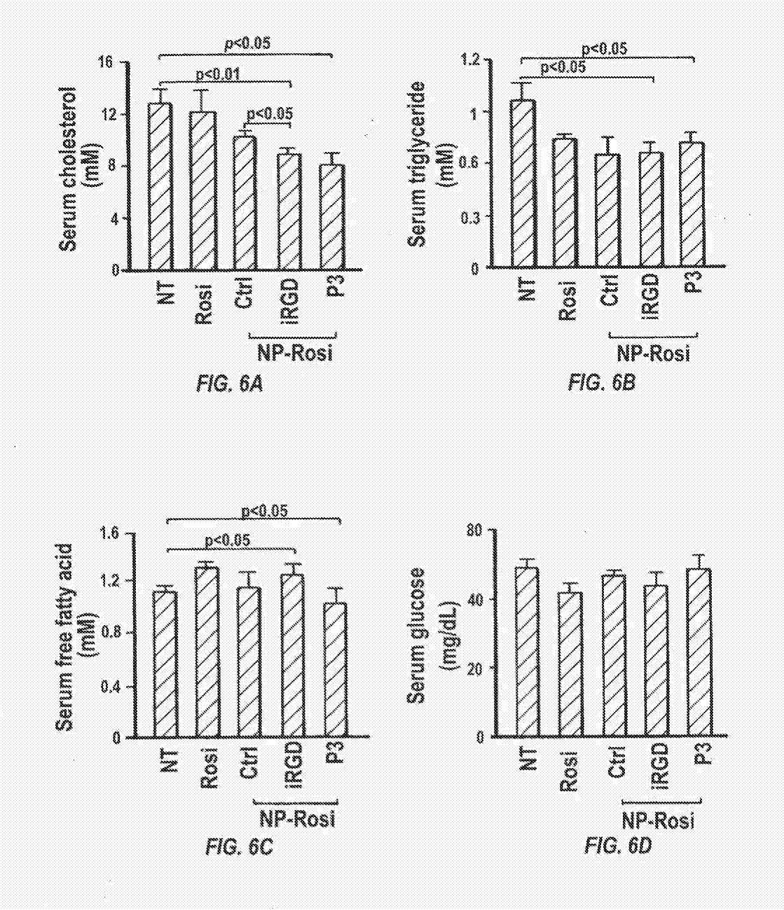

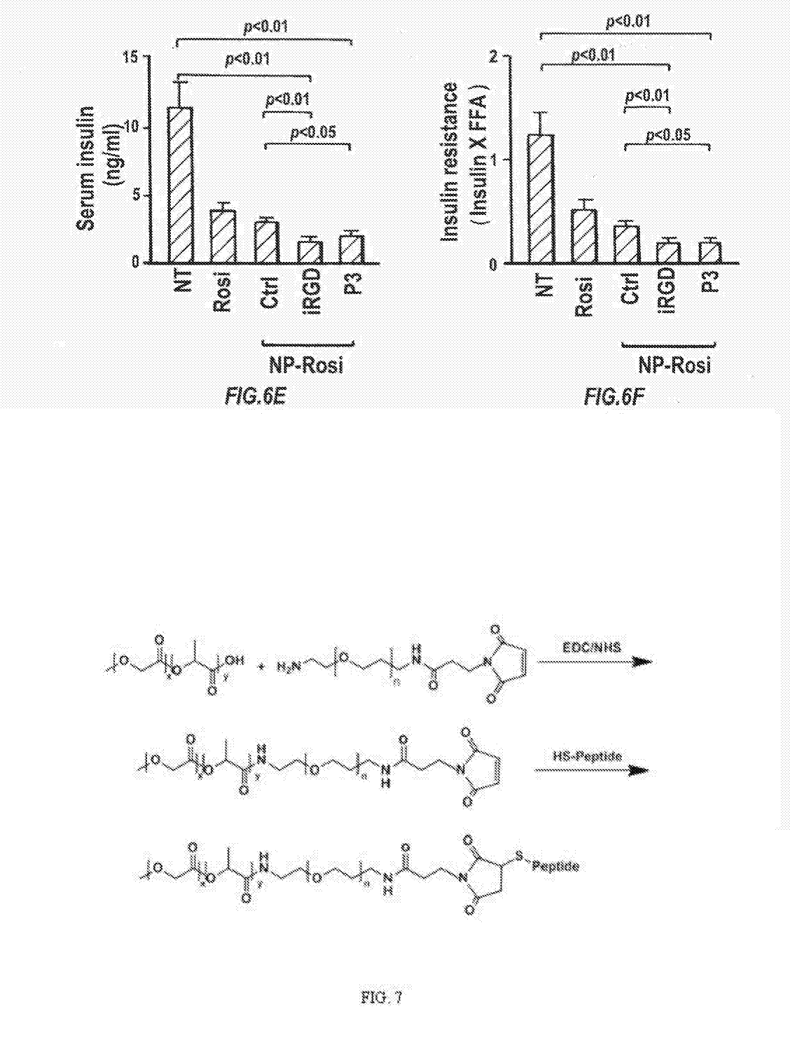

The nanoparticles can be used to lower serum cholesterol in subjects in need thereof. An effective amount of the nanoparticles loaded with a browning agent and targeted to WAT or WAT vasculature to promote angiogenesis and transformation of white adipose tissue into brown-like adipose can reduce serum cholesterol levels in the subject relative to an untreated control. FIG. 6A shows a significant reduction in serum cholesterol levels in mice treated with the nanoparticles. FIG. 6B shows that triglyceride levels were also reduced. Therefore, another embodiment provides a method for reducing serum triglyceride in a subject in need thereof by administering an effective amount of the nanoparticles loaded with a browning agent and targeted to WAT or WAT vasculature to reduce serum triglyceride levels in the subject.

E. Treatment of Inflammation

The compositions can be used to treat inflammation by administering to a subject in need thereof nanoparticles targeted to inflamed tissue. The nanoparticles can be loaded with anti-inflammatory drugs or chemotherapeutic agents as needed. The nanoparticles can also include a target inducing agent that induces expression of targets specific for inflamed or cancerous tissue. Representative targets for inflammation include, but are not limited to MECA-79 and DARC (Middleton et al., J. Pathol., 206(3):260-8 (2005)).

F. Dosage Regimens

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

In the preferred embodiments, the nanoparticles are administered by injection intravenously, intramuscularly, intraperitoneally, or subcutaneously.

The present invention will be further understood by reference to the following non-limiting examples.

### EXAMPLES

### Example 1

### Syntheses of Peptide-Conjugated NPs

### Materials and Methods

Reagents and Antibodies

N-hydroxysuccinimide (NHS), N,N-diisopropylethylamine (DIEA), Rosiglitazone, 1-ethyl-3-[3-dimethylaminopropyl]carbodiimide hydrochloride (EDC), triethylamine (Et₃N), and all other solvents and chemicals were purchased from Aldrich. 50:50 poly(DL-lactide-co-glycolide) (PLGA, inherent viscosity: 0.55-0.75 dL/g) with acid end groups was purchased from Adsorbable Polymers International. A PEG polymer of molecular weight 3,400 with a terminal amine and Maleimide group (NH₂-PEG-MAL) was purchased from JenKem Technology USA. Peptides were purchased from GL Biochem (Shanghai) Ltd. 16,16-dimethyl PGE2 was purchased from Santa Cruz Biotechnology Inc. Analytical Gel Permeation Chromatography (GPC) was performed using a Waters system equipped with a 2400 differential refractometer, 515 pump, and 717-plus autosampler. The flow rate was 1 ml min-1 and the mobile phase was tetrahydrofuran (THF). Linear polystyrene standards were used for calibration. A rat anti-mouse PECAM-1 (CD31) monoclonal antibody (BD Pharmingen, San Diego, USA), a biotinylated-isolectin B4 (Vector laboratories, Peterborough, UK), a rabbit anti-mouse UCP1 (Abcam, Cambridge, UK), an Alexa-555red-labeled goat anti-mouse IgG (Molecular Probes, CA, USA), an Alexa-555 red-labeled goat anti-rat IgG (Molecular Probes), a goat anti-rat IgG labeled with Alexa-488 (Molecular Probes) were used in the studies. ¹H NMR spectra were recorded on a Bruker AVANCE-400 NMR spectrometer. The NP sizes and 1-potentials were obtained by quasi-electric laser light scattering using a ZetaPALS dynamic light-scattering (DLS) detector (15 mW laser, incident beam 676 nm; Brookhaven Instruments). Transmission electron microscopy (TEM) was performed on a JEOL 2011 at 200 kV. The Agilent 1100 high performance liquid chromatography (HPLC, Santa Clara, Calif.) was equipped with a UV detector and a reverse-phase column (Eclipse, 4.6×150 mm, 5 μm).

Synthesis of PLGA-b-PEG-MAL

250 mg PLGA was dissolved in 2.5 mL dry dichloromethane (DCM) under magnetic stirring in a tightly sealed vial. Once the material was dissolved, 4.8 mg of EDC and 3 mg of NHS were added and the mixture was left to stir at room temperature (RT) for 90 min. The solution was then added dropwise into ice-cold mixture of diethyl ether and methanol and the resultant precipitate was centrifuged. Once the supernatant was decanted, the pellet was dissolved in DCM, and the precipitation/wash cycle was repeated three times before the activated PLGA-NHS ester was dried under vacuum. The yielded PLGA-NHS pellet was dissolved in dry chloroform. 20.4 mg of amine-PEG-maleimide and 2 μL of DIEA were added to the PLGA-NHS solution, followed by overnight stirring. The copolymer was precipitated with cold diethylether/methanol. The pellet was centrifuged and redissolved in DCM, followed by repeated precipitation/wash cycles (three times) to remove unreacted PEG. The resulting PLGA-b-PEG block copolymer was dried under vacuum and used for NP preparation. ¹H NMR (CDCl₃ at 400 Hz): δ 5.2 (m, —OCH(CH₃)C(O)NH—), 4.8 (m, —OCH₂C(O)O—), 3.7 (s, —CH₂CH₂O—), 1.6 (d, —OCH(CH₃)C(O)NH—) ppm. The resulting polymer was also characterized by GPC (Mw=56.84 kDa, PDI=1.38).

Synthesis of PLGA-b-PEG-Peptide Conjugates

For peptide conjugation, the PLGA-b-PEG-Mal diblock copolymer (200 mg) was dissolved in 1 mL of dry acetonitrile/DMF (50/50), and to this was added peptide (25 mg) with a terminal thiol group, followed by overnight stirring. The product was precipitated with ice-cold mixture of diethyl ether/methanol. The pellet was centrifuged and redissolved in DCM, followed by repeated precipitation/wash cycles (three times) to remove unreacted residues. The resulting PLGA-b-PEG-peptide was dried under vacuum. The molecular weight was characterized by GPC (Mw=59.38 kDa, PDI=1.33).

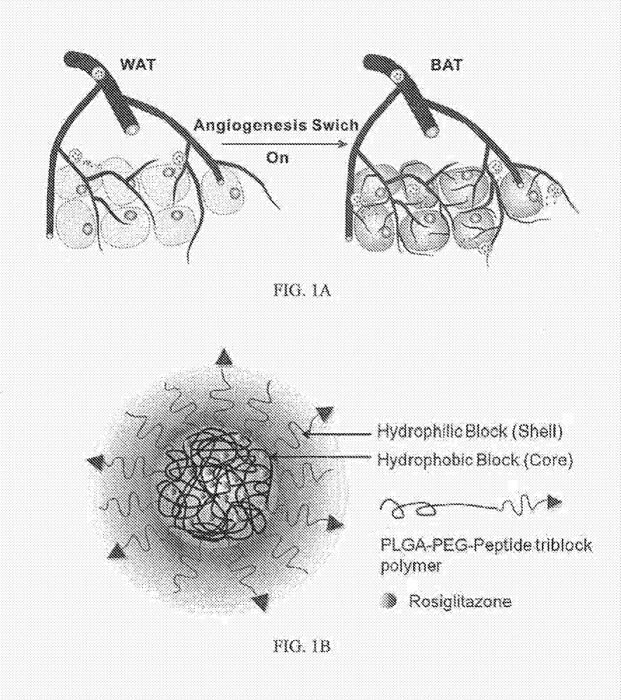

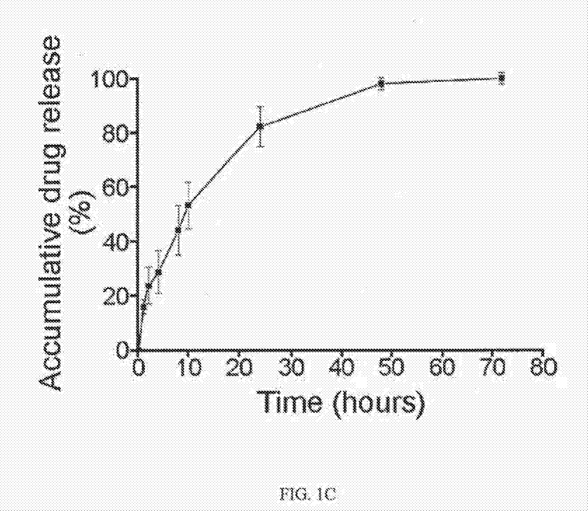

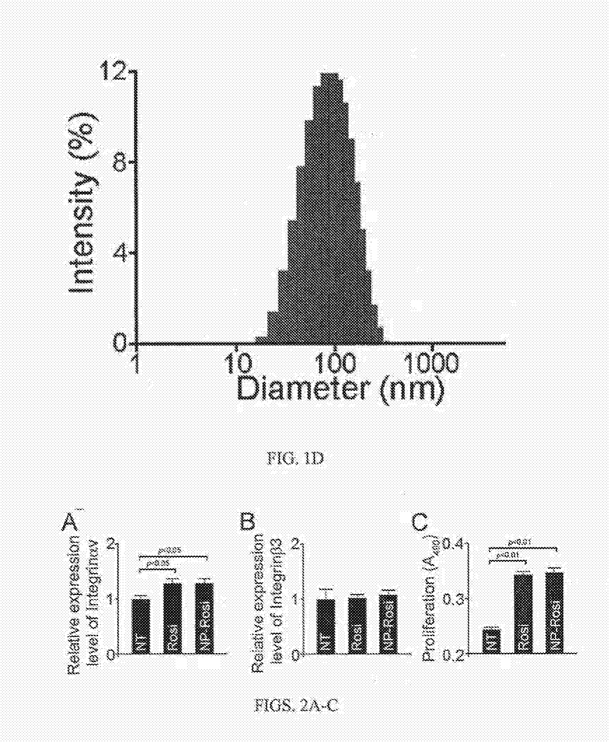

Two adipose vasculature-targeted peptides, iRGD or P3, were covalently conjugated to PLGA-PEG-MAL via the free thiols of the peptides' C-terminal using maleimide chemistry. The iRGD (CRGDK/RGPD/EC) peptide binds to angiogenic vessels through Integrinαvβ3/β5 receptors, and to home the drug-carrying cargo to local tissues. The P3 (CKGGRAKDC) peptide has been reported to specifically bind to WAT vasculature through the membrane protein prohibitin. Either Rosi or PGE2 was encapsulated within the peptide-PLGA-PEG-MAL NP using the emulsion solvent evaporation technique, and they are designated as iRGD-NP-Rosi or P3-NP-Rosi respectively (FIG. 1B and FIG. 7). Rosi and PGE2 have adipose tissue “browning” effects, which can induce adipose tissue transformation and angiogenesis. The NPs containing Rosi have an average size of 100 nm (FIG. 1D). The accumulative drug releasing profile showed that the NPs could continuously release drugs up to 40-50 hours, with the half-life of 12 hours (FIG. 1C).

### Example 2

### NPs Stimulate SVF In Vitro and Target Adipose Tissue In Vivo

### Methods and Materials

Animals, Tissue Collection and Histology

All animal studies were approved by the Committee on Animal Care, Massachusetts Institute of Technology. 6-week-old male wild-type mice and 10-week-old male diet-induced obese mice, of the C57Bl/6 strain, were used in current studies. High-fat-diet (TD-06414, 60/Fat) was purchased from Harlan Laboratories, and was used to induce and maintain obesity in C57Bl/6 mice.

The animals were euthanized by lethal dose of carbon-dioxide, and inguinal WAT, epididymal WAT, interscapular BAT and livers were immediately dissected out. For imaging, fluorescent pictures of tissues and organs were captured by IVIS imaging system (PerkinElmer, Waltham, Mass.). Portions of tissues were immersed into liquid nitrogen and were stored in −80 C until further use. Other portions of tissues were fixed in 4% paraformaldehyde (PFA) at 4 C overnight. For histological assessments, tissue samples were continued for performing whole mount staining or transferred into phosphate-buffered saline (PBS), followed by embedding in paraffin.

Isolation and Culture of the SVF from Adipose Tissues

WAT was dissected and extensively washed with Hank's Balanced Salt Solution (HBSS, Invitrogen). Tissues were cut into small pieces using a surgical scissor, and digested with 0.1% collagenase A in HBSS and 1% bovine serum albumin (SigmaAldrich) for 30 minutes at 37 C with gentle shaking. The digested tissues were suspended in Dulbecco's modified Eagle's medium containing 10% fetal bovine serum, and then passed through a 100 μm cell strainer (BD Falcon) to remove debris. Subsequently, cell suspension was centrifuged for 10 minutes at 300×g. 3. After removal of the floating cell layer and supernatant, the cell pellet was resuspended in 10 ml of HBSS, and then filtered through a 40 μm cell strainer (BD Falcon). The filtered cell suspension was layered on top of a Histopaque-1077 solution (10 ml) in a 50 ml tube, and then centrifuged at 400 g for 30 min at room temperature. The SVF cell layer at the gradient interface was collected and washed with HBSS. Cell counts and viability assessment were performed. Primary SVF or sorted cells were maintained in DMEM plus 10% FCS, 1% penicillin/streptomycin, and 10 ng/ml murine bFGF (R&D Systems).

Preparation and Characterization of NPs Containing Rosi or PGE2

The NPs encapsulated with Rosiglitazone or PGE2 was formulated via emulsion solvent evaporation technique. In brief, copolymers PLGA-b-PEG and PLGA-b-PEG-Peptide (20:1) were dissolved in DCM with or without respective drug compound. The polymer/drug solution (1 mL) was added into 3 mL aqueous solution containing 1% PVA, followed by probe sonification to form the emulsion. The emulsified mixture was poured into 15 mL water and stirred for 2 hours to allow the DCM solvent to evaporate. The remaining organic solvent and free molecules were removed by washing the particle solution three times using an Amicon Ultra-4 centrifugal filter (Millipore, Billerica, Mass.) with a molecular weight cutoff of 100 kDa. The NP size and zeta potential were determined by using DLS. Samples for TEM were stained with 1% uranyl acetate and observed using a JEOL 2011 at 200 kV. The drug content in the NPs was analyzed by RP-HPLC (Agilent, CA). Drug loading is defined as the mass fraction of drug in the NPs, whereas entrapment efficiency (EE) is the fraction of initial drug that is encapsulated by the NPs.

Release of Rosi from the NPs

To determine the release kinetics, a suspension of Rodi-loaded NPs in phosphate buffered saline (PBS) was aliquoted (100 μL) into semi-permeable minidialysis tubes (molecular weight cutoff 100 kDa; Pierce) and dialyzed against frequently renewed PBS (pH 7.4) at 37° C. with gentle stirring. At a predetermined time, quadruplicate aliquots of each NP suspension were withdrawn for RP-HPLC analysis. Rosi content was quantified by detecting the absorbance at 254 nm. The mobile phase consists of ammonium acetate (10 nM, pH 5.2) and acetonitrile (60:40 V/V) with a flow rate of 1 ml/min.

Treatment with Free Drug and NPs

Standard diet fed or high-fat-diet fed C57Bl/6 mice were intravenous injected with either NP constructs or free drug (rosiglitazone or 16,16-dimethyl-PGE2) at the drug dose of 80 mg/kg, every second day. Free Rosi was dissolved in DMSO followed by dilution in saline.

Statistical Analyses

All experiments were repeated at least twice. Statistical analyses on the in vitro and in vivo results were made by a standard two-tailed unpaired Student's t-test using Microsoft EXCEL 2010.

### Results

Angiogenic vessels usually express high levels of integrins, particularly αv and β3 subunits. To evaluate whether Rosi-loaded NPs can effectively induce expression of integrins on vascular cells, the stromal vascular fragments (SVF) were isolated from WAT, followed by treatment with Rosi (1 μM) in either solution or non-targeted NP form. CD31+ SVF was sorted and stained with Integrinαv antibody, Isolectin B4 and DAPI. C57Bl/6 mice were treated with Rosi, NP-Rosi, iRGD-NP-Rosi or P3-NP-Rosi for two weeks (n=3). IngWAT, epiWAT and livers were dissected and representative samples were imaged under IVIS imaging system. Inguinal WAT and epididymal WAT were dissected and representative samples were photographed.

Quantitative real-time PCR (qRT-PCR) assay revealed that free Rosi and Rosi-loaded NPs elevated the expression levels of Integrinαv, but not Integrinβ3 on SVF in vitro (FIGS. 2A and B).

Furthermore, treatment with either Rosi or NP-Rosi stimulated the proliferation of SVF. It should also be noted that no evidence of cellular toxicity was observed under all the conditions. SVFs were sorted with an endothelial marker CD31, and stained cells with an Integrinαv antibody. Integrinαv was upregulated by Rosi and NP-Rosi treatments. These results confirm that Rosi stimulates angiogenesis in vitro both in solution and NP forms.

To illustrate the distribution of peptide-NP constructs in vivo, fluorescent signal from NPs were analyzed using the IVIS imaging system following tail vein injection of FAM-labeled iRGD-NP-Rosi or P3-NP-Rosi into mice. One hour after the infusion, FAM-labeled iRGD-NP-Rosi and P3-NP-Rosi accumulated in inguinal and epididymal WATs, though there was non-specific accumulation in the livers of all treatment groups due to active liver metabolism. Only minimal accumulation of non-targeted NP (Ctrl) was observed in WAT. These results suggested that iRGD and P3 peptides could facilitate NP homing into WATs in vivo.

### Example 3

### NPs Stimulate Angiogenesis and Facilitate Adipose Tissue Transformation In Vivo

### Materials and Methods

Animals used in this example are described above. NPs used in the experiments are described above.

Immunohistochemistry

The PFA-fixed tissues were stained as previously described (Xue, et. al., Nature Medicine, 2012, 18:100-110) Briefly, tissue samples were digested with 20 mg/ml proteinase K for 5 minutes, followed by incubation in methanol for 30 minutes. The permeabilized tissues were stained with a rat anti-mouse CD31 antibody (1:300 dilution) at 4° C. overnight, and Alexa 555-conjugated anti-rat secondary antibody (1:500 dilution) at room temperature for 2 hours, respectively. Between each step, rigorous washing in PBS was required. Samples were mounted in Vectashield mounting medium (Vector Laboratories, Inc., Burlingame, Calif., USA) and stored at −20° C. in the dark before examination under a confocal microscope (Zeiss Confocal LSM710 Microscope. Images were further analyzed with the Adobe Photoshop CS software program.

Paraffin-embedded tissues were sectioned into 5 mm thick slides. Tissue slides were stained with anti-isolectin B4 antibody (1:500) or anti-UCP1 antibody (1:200) according to a standard Avidin-Biotin-Complex protocol. DAB was used as a chromogen to illustrate the positive staining.

### Result

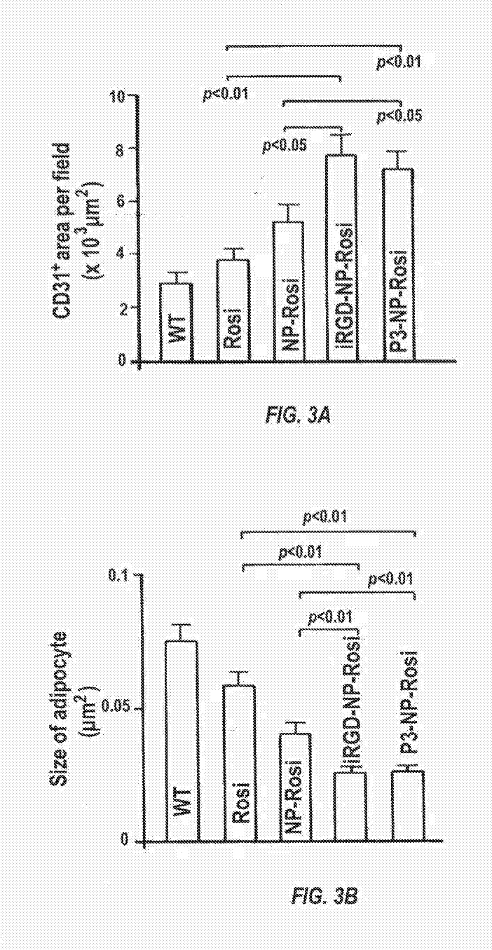

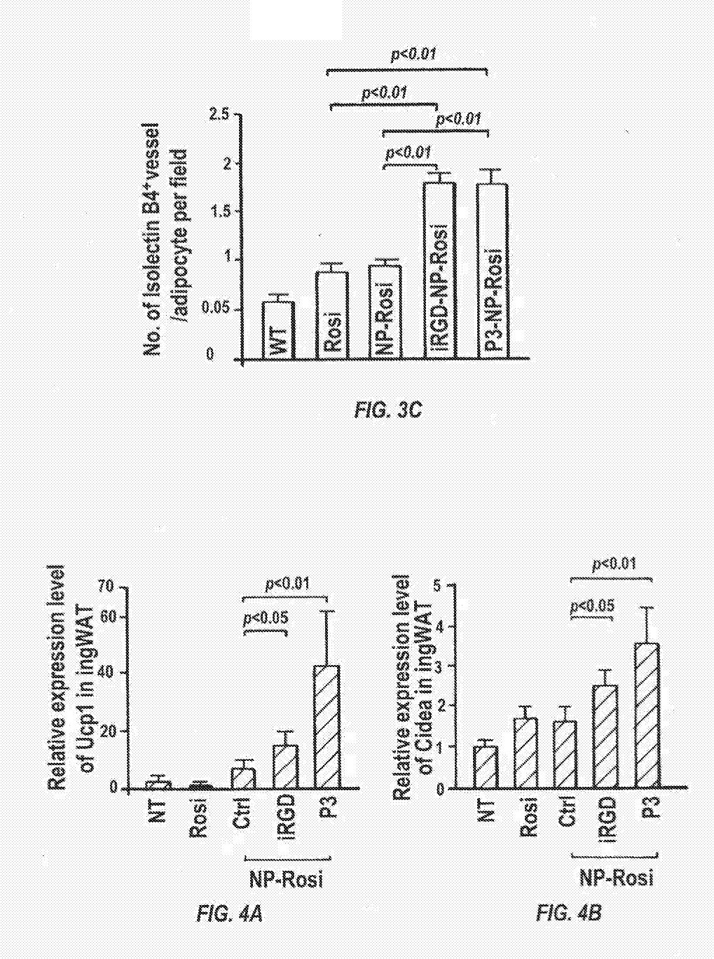

Wild-type C57Bl/6 mice were treated with free Rosi, NP-Rosi, iRGD-NP-Rosi and P3-NP-Rosi. Fifteen days post treatment, complete necropsy was conducted to determine the phenotypical changes in various fat tissues at inguinal and epididymal areas after euthanizing the mice. Both inguinal and epididymal WATs from mice treated with iRGD-NP-Rosi and P3-NP-Rosi appeared more reddish color compared with control groups, suggesting more intensive vascularization in WATs and possible increased cellular contents in adipocytes. On the cellular level, general histological staining on inguinal WAT with hematoxylin and eosin indicated the shrinkage of adipocytes, condensed cellular contents and transformation of adipocytes (FIG. 3B). Groups treated with iRGD-NP-Rosi and P3-NP-Rosi showed elevated expression of BAT marker UCP1 on inguinal WAT compared to control groups (data not shown). Furthermore, immuno-staining against EC specific marker CD31 illustrated the increased density of blood vasculature in inguinal WAT of mice treated with iRGD-NP-Rosi and P3-NP-Rosi (FIG. 3A). Additionally, the staining that used an independent EC marker Isolectin B4 is consistent with these results. Since the variable size of adipocytes could affect the vascular density, the vessel numbers are normalized against adipocyte numbers per optical field and found that the blood vessel numbers were indeed increased by more than 3 folds post iRGD-NP-Rosi and P3-NP-Rosi treatments (FIG. 3C). In epididymal WAT, shrinkage of adipocyte size and increase of blood vessels were induced by iRGD- and P3-NP-Rosi treatments (data not shown). Taken together, these results demonstrate that iRGD-NP-Rosi and P3-NP-Rosi lead to higher vascular density in comparison to nontargeted NP and free drug control groups, therefore greatly facilitating WAT transformation into brown-like adipose tissue in mice.

### Example 4

### NPs Upregulate BAT and EC Markers in WATs

### Materials and Methods

Quantitative Real-Time PCR

Adipose tissues were homogenized in Ultraspec (Biotecx, Houston, Tex.) and total RNA was isolated with Lipid Tissue RNA Isolation Kit (Qiagen, Duesseldorf Germany). Quantitative real-time PCR was performed following the TaqMan RNA-to-Ct One-Step method with the primers and probes designed by Applied Biosystems. Detection of mRNA was performed using a StepOnePlus Sequence Detection system (Applied Biosystems). The mean of the values obtained in non-treated animals was set to 100% and values obtained in other groups of animals were expressed relative to this.

### Results

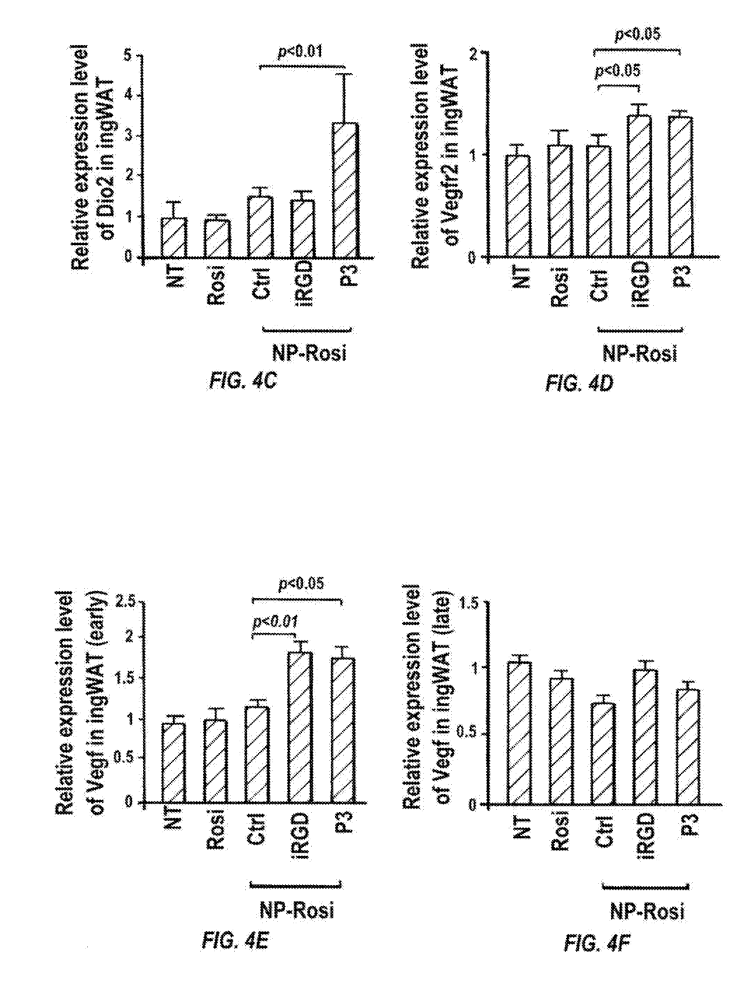

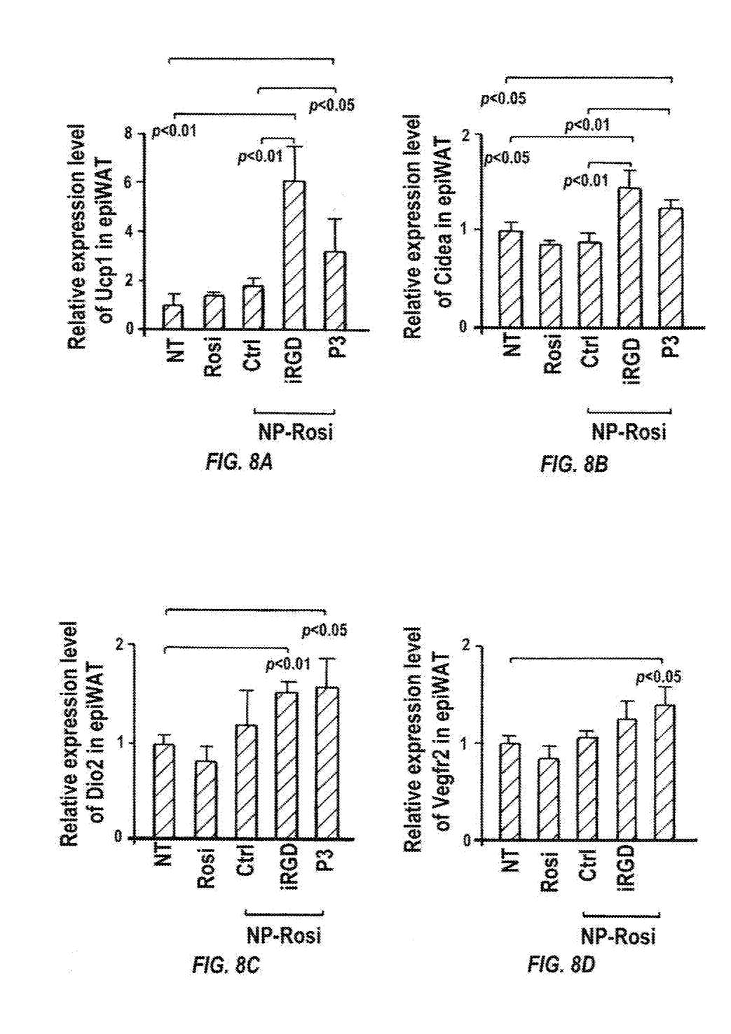

qRT-PCR was used to investigate whether the expression patterns of various BAT and EC markers will be altered on the molecular level by NPs treatment. In inguinal WAT, UCP1 expression level was significantly upregulated by treatments with iRGD-NP-Rosi and P3-NP-Rosi (FIG. 4A). A marked induction in the expression of another BAT marker, transcriptional coactivator CIDEA, was also observed post treatment with the two targeted NPs (FIG. 4B). Notably, the treatment with P3-NP-Rosi induced type 2 deiodinase (Dio2) expression, indicating an active brown fat phenotype (FIG. 4C). Vascular EC marker, VEGF receptor 2 (VEGFR2), was also upregulated by the treatment with the two peptide-conjugated NPs (FIG. 4D). Intriguingly, the expression of VEGF was upregulated in the early stage (4 days) but not in the late stage of treatment (15 days) (FIGS. 4E and F). Similarly, iRGD-NP-Rosi and P3-NP-Rosi treatments induced higher expression levels of UCP1, CIDEA, and DIO2 in epididymal WAT (FIG. 8A-D).

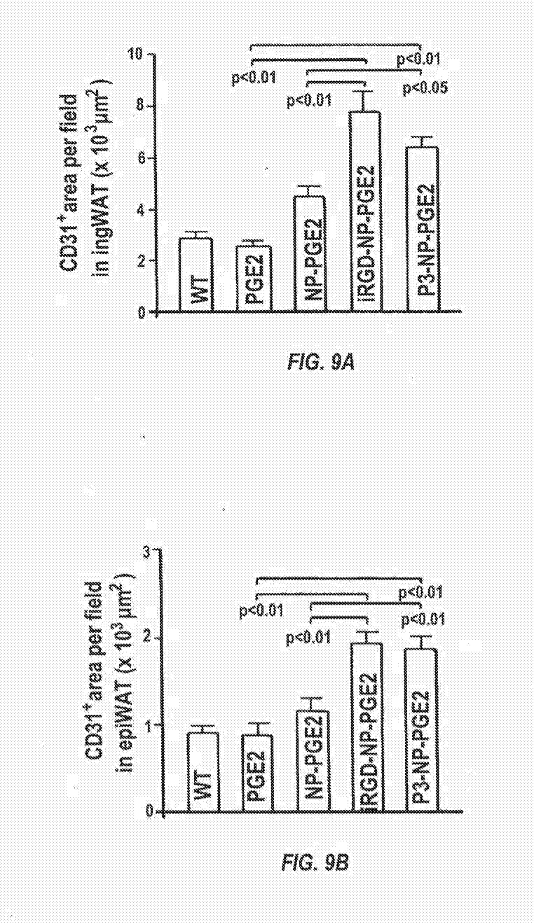

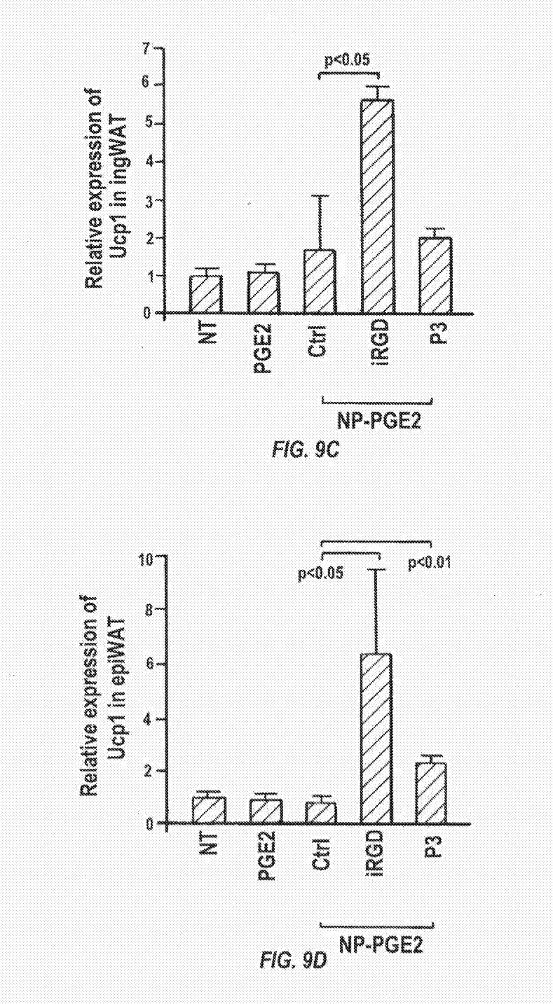

To evaluate the potential of applying this amplified drug delivery system to other drugs, mice were treated with PGE2 loaded NPs to observe angiogenesis stimulation and WAT transformation. Similar to Rosi loaded NPs, iRGD-NP-PGE2 and P3-NP-PGE2 treatments increased the density of CD31 positive blood vasculature by about 2-fold in both inguinal and epididymal WAT (FIG. 9A-B). The expression of UCP1 was also upregulated by those treatments (FIGS. 9E and D). The positive results suggest promising potential for the treatment of obesity with a wide spectrum of encapsulated drugs using this nanomedicine platform exhibiting amplified drug delivery characteristics.

### Example 5

### Enhanced Effect of Targeted NPs on Inhibition of Obesity Development in DIO Mice

### Materials and Methods

Animals used in this example are described above. NPs used in the experiments are described above.

### Results

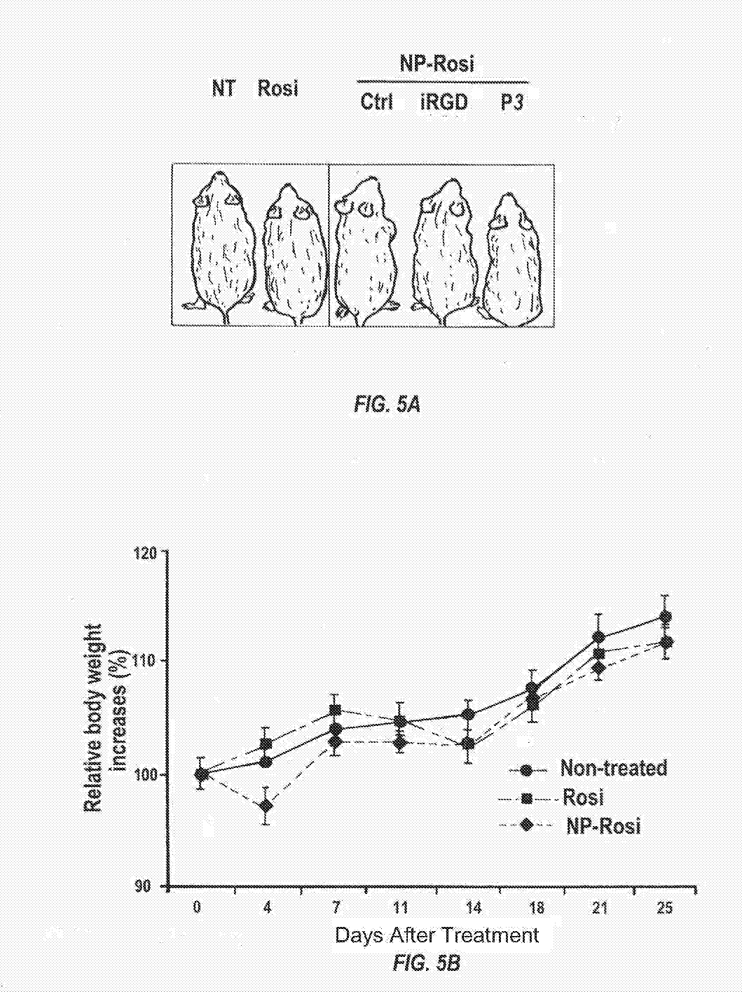

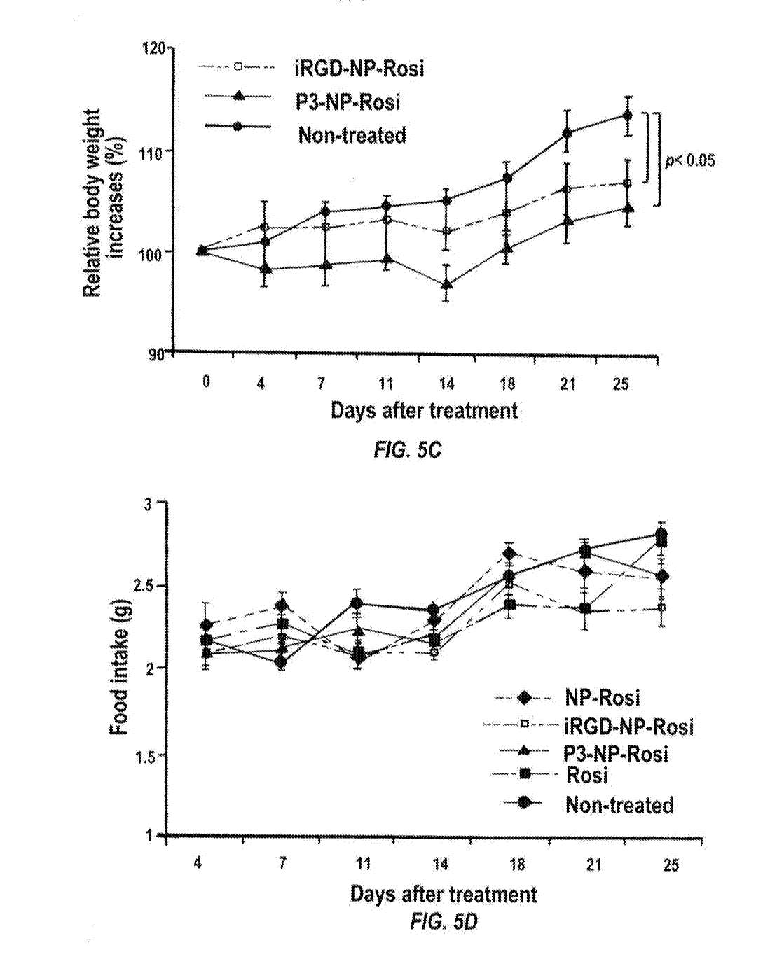

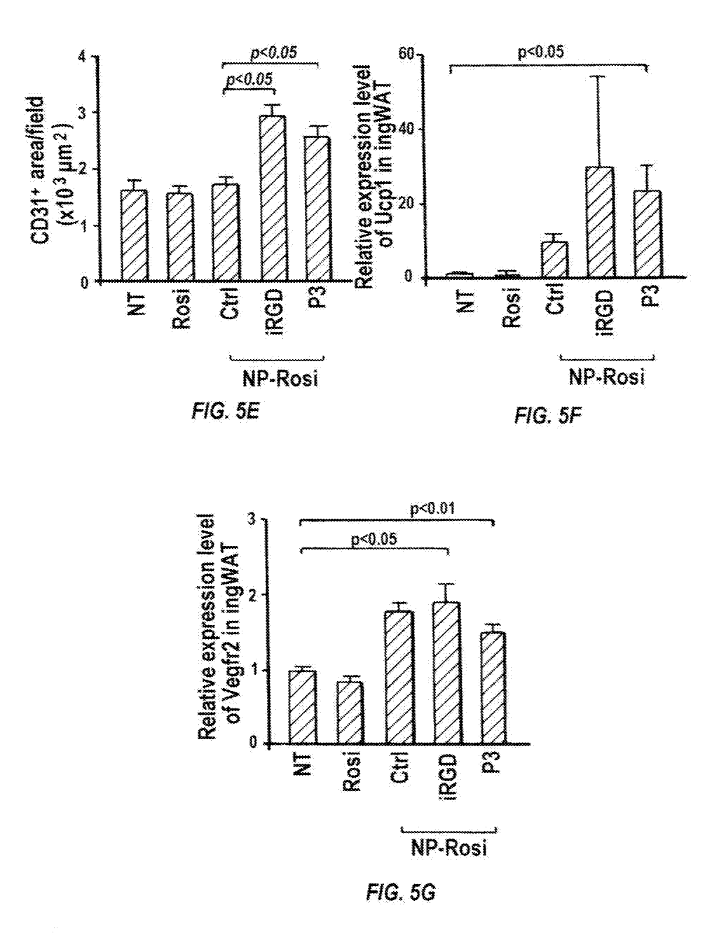

Since adipose tissue transformation and angiogenesis could be initiated by treatment with the two peptide-NP-Rosi constructs, whether such treatment could regulate the metabolism, inhibit the development of obesity, and demonstrate any therapeutic effects in obese mice was investigated. The high-fat-diet induced obese model was used to assess the possible impact of the two peptide-NP-Rosi on body weight, tissue histology, and molecular expression profiles. There was no difference in body weight gain between the mice treated with Rosi (in solution and non-targeted NP forms) and non-treated obese mice (FIGS. 5A and B). In contrast, both targeted NPs were able to significantly inhibit the body weight gain in DIO mice 25 days post treatment (FIGS. 5A and C). It was worthy to note that mice from different treatment groups demonstrated similar food intakes (FIG. 5 D). H&E staining exhibited the shrinkage of adipocytes in WAT by both targeted NP treatments. CD31 positive adipose tissue vasculature were markedly increased about 2-fold in mice receiving iRGD-NP-Rosi and P3-NP-Rosi treatments compared with control groups (FIG. 5E). Meanwhile molecular expression levels of UCP1 and VEGFR2 were upregulated by both peptide-functionalized NP treatments (FIGS. 5F and G). These findings reveal that both iRGD-NP-Rosi and P3-NP-Rosi exhibited significant anti-obesity effects in a DIO mouse model by activating adipose tissue transformation and angiogenesis.

### Example 6

### Lipid and Carbohydrate Metabolism in DIO Mice

### Materials and Methods

Animals used in this example are described above. NPs used in the experiments are described above.

Assessments on Serological Parameters

Serum levels of free fatty acid, glucose, triglycerides and cholesterol contents from fasted obese mice were determined by standard kits from Cayman Chemical Co. (Ann Arbor, Mich.). Serum insulin from fasted obese mice was determined by ELISA method according to manufacturer's protocol (Millipore, Billerica, Mass.).

### Results

Serum was collected from DIO mice that were fasted for five hours following 25 days of treatments, and analyzed for levels of serological parameters. As shown in FIG. 6A, DIO mice receiving either iRGD-NP-Rosi or P3-NP-Rosi showed a significant 30% reduction in serum cholesterol compared to non-treated group. Furthermore, both iRGD-NP-Rosi and P3-NP-Rosi treatments resulted in statistically (p<0.05) marked decrease in serum triglyceride level when compared to non-treatment (NT) group (FIG. 6B). Importantly, there was no significant difference in the levels of serum free fatty acid (FFA) and glucose among all groups (FIGS. 6C and D). However, free Rosi and non-targeted version of NP reduced the production of serum insulin by 3-folds, while iRGD-NP-Rosi and P3-NP-Rosi resulted in even greater serum insulin reduction (FIG. 6E). We also calculated the indirect insulin resistance index (IR=Insulin×FFA) which was determined to be the lowest in the mice receiving both targeted NP treatments among all the tested groups as shown in FIG. 6F, indicating elevated insulin sensitivity.

1. A nanoparticle comprising
a targeting moiety specifically binding to a target molecule on adipose or organ tissue;
a target molecule inducing agent inducing expression or bioavailability of the target molecule in the subject to promote additional nanoparticles to bind to the induced target molecule.

2. The nanoparticle of claim 1 further comprising agent to be released at the site of binding selected from the group consisting of therapeutic, prophylactic and diagnostic agents.

3. The nanoparticle of claim 1 comprising polymer, lipid, inorganic molecules or combination thereof.

4. The nanoparticle of claim 1 comprising
a hydrophilic outer shell,
a targeting moiety specifically binding to a target molecule on adipose or organ tissue; and
a hydrophobic inner core.

5. The nanoparticle of claim 2 wherein the nanoparticle binds to the target molecule when administered to a subject and releases therapeutic, prophylactic or diagnostic agent.

6. The nanoparticle of claim 2 wherein the target molecule inducing agent induces the expression or bioavailability of the target molecule in the subject to promote additional nanoparticles to bind to the induced target molecule and thereby increase the local concentration of the agent to be released.

7. The nanoparticle of claim 1, wherein the target inducing agent comprises an adipose tissue browning agent.

8. The nanoparticle of claim 7, wherein the adipose tissue browning agent is selected from the group consisting of a peroxisome proliferator activated receptor gamma (PPARγ) activator, a prostaglandin E2 analog, and a combination thereof.

9. The nanoparticle of claim 8, wherein the PPARγ activator comprise rosiglitazone.

10. The nanoparticle of claim 8, wherein the prostaglandin E2 analog comprises (16,16-dimethyl PGE2, PGE2).

11. The nanoparticle of claim 1, wherein the targeting moiety comprises an iRGD peptide or a P3 peptide.

12. The nanoparticle of claim 11, wherein the iRGD peptide comprises the amino acid sequence CRGDK/RGPD/EC and the P3 peptide comprises the amino acid sequence CKGGRAKDC.

13. The nanoparticle of claim 1 comprising an outer polyethylene glycol surface with a targeting peptide and a polylactide-co-glycolide core containing Rosiglitazone.

14. The nanoparticle of claim 1, wherein the target inducing agent is released over time.

15. The nanoparticle according to claim 1 further comprising a detectable label.

16. The nanoparticle according to claim 15, wherein the detectable label is selected from the group consisting of a fluorescent label and a contrast agent.

17. The nanoparticle of claim 1 comprising
a hydrophobic copolymer core comprising an adipose tissue browning agent to increase vascularization and adipose tissue transformation; and
a hydrophilic polymer corona surrounding the hydrophobic copolymer core, wherein the hydrophilic polymer corona comprises a vasculature targeting moiety that targets the nanoparticle to adipose tissue angiogenic vessels to facilitate homing of the nanoparticle to white adipose tissue and increase angiogenesis in the white adipose tissue to amplify delivery of additional nanoparticles to the white adipose tissue.

18. A pharmaceutical composition comprising the nanoparticles according to claim 1.

19. A dosage formulation comprising nanoparticles of claim 8, comprising nanoparticles delivering PPARγ activator in an amount effective to induce elevated expression levels of VEGF and angiopoietin-like 4 in adipose tissue relative to untreated adipose tissue.

20. The dosage formulation of claim 19, wherein the PPARγ activator is present in an amount effective to increase expression of Integrinαv on vascular cells of adipose tissue.

21. The dosage formulation of claim 20, wherein the vascular cells comprise endothelial cells.

22. A method for treating obesity in a subject in need thereof, comprising the steps of
administering to the subject the nanoparticle according to claim 1 in an amount effective to promote angiogenesis and transformation of white adipose tissue into brown-like adipose, wherein the transformation of white adipose tissue to brown-like adipose tissue increases weight loss over time

23. A method of transforming white adipose tissue to brown-like adipose tissue in a subject in need thereof comprising:
administering to the subject an effective amount of a nanoparticle according to claim 1 to promote angiogenesis and transformation of white adipose tissue into brown-like adipose.

24. A method of reducing serum cholesterol levels in a subject in need thereof, comprising:
administering to the subject an effective amount of a nanoparticle according to claim 1 promote angiogenesis and transformation of white adipose tissue into brown-like adipose to reduce serum cholesterol levels in the subject relative to an untreated control.

25. A method for treating a metabolic disorder in a subject in need thereof comprising,
administering to the subject an effective amount of a nanoparticle according to claim 1 to promote angiogenesis and transformation of white adipose tissue into brown-like adipose to treat one or more symptoms of the metabolic disorder.

26. The method of claim 25, wherein the metabolic disorder is selected from the group consisting of obesity, insulin resistance, hyperinsulinemia, hypoinsulinemia, type II diabetes, hypertension, hyperhepatosteatosis, hyperuricemia, fatty liver, non-alcoholic fatty liver disease, polycystic ovarian syndrome, acanthosis nigricans, hyperphagia, endocrine abnormalities, triglyceride storage disease, Bardet-Biedl syndrome, Lawrence-Moon syndrome, Prader-Labhart-Willi syndrome, and muscle hypoplasia.
